SEQUENCE LISTING (28 sequences)

SEQ ID NO: 1 - DNA, 52 bp, Homo sapiens
  MISC_FEATURE (27)..(27): n = g or t
ttacagtggtcggtcacaagaaaccanctgaacaatttcagtcatttgaagc

SEQ ID NO: 2 - DNA, 52 bp, Homo sapiens
  MISC_FEATURE (27)..(27): n = c or t
cccaaatgatacagatactacacttanacattaccatgtcagtattcactga

SEQ ID NO: 3 - DNA, 52 bp, Homo sapiens
  MISC_FEATURE (27)..(27): n = c or t
cctactgaaggcctgtgtattattgtntttctccttttggtttatccacatt

SEQ ID NO: 4 - DNA, 52 bp, Homo sapiens
  MISC_FEATURE (27)..(27): n = c or g
gtaatgactcaaataatagttctcaantccagaagcccactatagtctacaa

SEQ ID NO: 5 - DNA, 52 bp, Homo sapiens
  MISC_FEATURE (27)..(27): n = c or t
gtgtgtgggctatagtttattgatccntgaacctaatgcataattacattta

SEQ ID NO: 6 - DNA, 52 bp, Homo sapiens
  MISC_FEATURE (27)..(27): n = c or t
aaattatatctaaatatgaaaaaagtnctaaagctaacatgttttaagttta

SEQ ID NO: 7 - DNA, 52 bp, Homo sapiens
  MISC_FEATURE (27)..(27): n = a or c
tatcggttctctttgtaataattaaanttgtcatatacatttgttttttatt

SEQ ID NO: 8 - DNA, 52 bp, Homo sapiens
  MISC_FEATURE (27)..(27): n = a or g
cttctattattaatggttctactttgntaacccttttattattgtgaagctt

SEQ ID NO: 9 - DNA, 52 bp, Homo sapiens
  MISC_FEATURE (27)..(27): n = a or g
aaaatacattttcccagaggttcattncaatttttggccaaactgagttgtt

SEQ ID NO: 10 - DNA, 52 bp, Homo sapiens
  MISC_FEATURE (27)..(27): n = c or g
aaaaggaatacagggaggagtgaagangccagaggactgtcaggttccatta

SEQ ID NO: 11 - DNA, 52 bp, Homo sapiens
  MISC_FEATURE (27)..(27): n = a or g
caatgggggagactcccagggcaacancaattaggaggaagggaaagtaaat

SEQ ID NO: 12 - DNA, 52 bp, Homo sapiens
  MISC_FEATURE (27)..(27): n = c or t
gcccttaggtgtgaagccttaagaaanggatgctttgagtcccagggctgag

SEQ ID NO: 13 - DNA, 52 bp, Homo sapiens
  MISC_FEATURE (27)..(27): n = a or g
ctatcctatatgaagtggtttggtgangggtcttgcttttatttgaattttt

SEQ ID NO: 14 - DNA, 52 bp, Homo sapiens
  MISC_FEATURE (27)..(27): n = c or t
gggaggcatctcaaggattctccttcnaggggatgaaatggagctcaaggaa

SEQ ID NO: 15 - DNA, 51 bp, Homo sapiens
  MISC_FEATURE (27)..(27): n = a or g
catctctcgggctcctggcccaggctntgtcttgttcccagcgctgagggc

SEQ ID NO: 16 - DNA, 52 bp, Homo sapiens
  MISC_FEATURE (27)..(27): n = c or g
aacagcctcctgttgggcaatttcctnttccagaatcaactccactacccat

SEQ ID NO: 17 - DNA, 52 bp, Homo sapiens
  MISC_FEATURE (27)..(27): n = a or g
tctgcttaagattgtttctagcatacnttatttcaatttaggcaaatgtgac

SEQ ID NO: 18 - DNA, 52 bp, Homo sapiens
  MISC_FEATURE (27)..(27): n = a or g
aataccaaaaaatatcaattttctgtngcattaccagccattaggcttaatt

SEQ ID NO: 19 - DNA, 52 bp, Homo sapiens
  MISC_FEATURE (27)..(27): n = c or t
tttgaaagtaatgatttgggggtaatncctttatgcttggggtgccaaggca

SEQ ID NO: 20 - DNA, 52 bp, Homo sapiens
  MISC_FEATURE (27)..(27): n = c or t
gcatttctacaaaggaattactagtgncaaacgctttttgctaggtcaaaca

SEQ ID NO: 21 - DNA, 52 bp, Homo sapiens
  MISC_FEATURE (27)..(27): n = a or g
ttcctcaaggaccagtaagatacaacnttagtcacactaattcccaactttg

SEQ ID NO: 22 - DNA, 52 bp, Homo sapiens
  MISC_FEATURE (27)..(27): n = a or g
actctggaacctgtggtattagatacnaagaattatctaattcaaggcagac

SEQ ID NO: 23 - DNA, 52 bp, Homo sapiens
  MISC_FEATURE (27)..(27): n = a or t
cgtttgagtgaaacaggatgcaatttncaagcaagggtaagtctcatccaat

SEQ ID NO: 24 - DNA, 52 bp, Homo sapiens
  MISC_FEATURE (27)..(27): n = c or t
tcctgaagcaggacccatgcaactcanaatgttctatggcagcatttgaaga

SEQ ID NO: 25 - DNA, 52 bp, Homo sapiens
  MISC_FEATURE (27)..(27): n = a or g
cataacactcaggtaattttaaagtgntaacaaatgactcttcatttcaaaa

SEQ ID NO: 26 - DNA, 52 bp, Homo sapiens
  MISC_FEATURE (27)..(27): n = c or t
aatgcaattcctagatgcagtatatanaagcatttttgcctagactaagtaa

SEQ ID NO: 27 - DNA, 52 bp, Homo sapiens
  MISC_FEATURE (27)..(27): n = c or g
aggtcttgaattcacagtgggaaagangaaaggcagcatggtttagatgctt

SEQ ID NO: 28 - DNA, 52 bp, Homo sapiens
  MISC_FEATURE (27)..(27): n = a or g
gtgatatttataaagatgtgatgactngtggtgatttcaatacacgaggaaa

(19) United States
(12) Patent Application Publication
(10) Pub. No.: US 2011/0177509 A1
(43) Pub. Date: Jul. 21, 2011

(54) RISK FACTORS AND A THERAPEUTIC TARGET FOR NEURODEGENERATIVE DISORDERS

(75) Inventors: Alison Goate, St. Louis, MO (US); Carlos Cruchaga, St. Louis, MO (US); David Holtzman, St. Louis, MO (US); Anna Fagan Niven, St. Louis, MO (US)

(73) Assignee: THE WASHINGTON UNIVERSITY, St. Louis, MO (US)

(21) Appl. No.: 13/055,569

(22) PCT Filed: Jul. 10, 2009
(86) PCT No.: PCT/US09/50255

§ 371 Date: Apr. 8, 2011

Related U.S. Application Data
(60) Provisional application No. 61/083,056, filed on Jul. 23, 2008.

(51) Int. Cl. C12Q 1/68; C07H 21/00; C40B 40/06

(52) U.S. Cl. 435/6.11; 536/23.5; 536/23.2; 536/24.31; 506/16

(57) ABSTRACT

Compositions and methods for detecting a neurodegenerative disorder, and methods of treating a neurogenerative disorder are disclosed. Biomarkers for a neurodegenerative disorder containing a polymorphism in the nucleotide sequence of PP3R1, GSK3beta, PPP3CA, FYN, WISP1, MGEA5, CTSD, F2, MAPT, OGT or PRKCA are also disclosed. A method for detecting a neurodegenerative disorder by detecting polymorphisms in the above genes is further disclosed.

### GOVERNMENTAL RIGHTS

This invention was made with government support under P50-AG05681, P01-AG03991, P01-AG026276, and R01-AG16208 awarded by the National Institute of Aging. The government has certain rights in the invention.

### FIELD OF THE INVENTION

The present invention encompasses compositions, methods for detecting a neurodegenerative disorder, and methods of treating a neurogenerative disorder.

### REFERENCE TO SEQUENCE LISTING

A paper copy of the sequence listing and a computer readable form of the same sequence listing are appended below and herein incorporated by reference. The information recorded in computer readable form is identical to the written sequence listing, according to 37 C.F.R. 1.821 (f).

### BACKGROUND OF THE INVENTION

The neuropathological hallmarks of Alzheimer's Disease (AD) are the presence of senile plaques (SP) and neurofibrillary tangles (NFTs) in brain (1). NFTs are intracellular deposits of abnormally hyperphosphorylated microtubule-associated protein tau (MAPT). Tau protein is located in axons and interacts with microtubules to promote their polymerization and stabilization (3). Tau activity depends on its state of phosphorylation (3), which is regulated by several kinases, phosphatases and other tau-related proteins (4). Hyperphosphorylation of tau destabilizes the microtubule network, leading to impaired axonal transport and ultimately to NTF formation and neuronal death (5).

The CSF levels of total tau and tau phosphorylated at threonine 181 (ptau₁₈₁) are increased in AD (9, 11). Elevated CSF tau levels are associated with neuronal damage and are also observed in stroke (12) and traumatic brain injury immediately after injury (13), however increases in CSF ptau₁₈₁ levels are only found in AD (14-16). The increase in CSF ptau₁₈₁ levels, when combined with CSF Aβ42, is a useful biomarker to predict cognitive decline from cognitively normal to mild cognitive impairment (9) and predicts decline in subjects with mild cognitive impairment and conversion to AD (14, 17, 18).

Because of the important role tau plays in the pathogenesis of neurodegenerative disorders, there is a need in the art for identifying the genetic source of the increased tau levels observed in these disorders. Identifying the genetic source will help to identify subjects at risk for a neurodegenerative disorder, such as AD, and may help provide targets for treating such a disorder.

### SUMMARY OF THE INVENTION

One aspect of the present invention encompasses a biomarker for a neurodegenerative disorder. The biomarker comprises at least one polymorphism in a nucleotide sequence selected from the group consisting of PPP3R1, GSK3β, PPP3CA, FYN, WISP1, MGEA5, CTSD, F2, MAPT, OGT and PRKCA. The polymorphism shows linkage disequilibrium and has a correlation value of greater than about 0.7 when compared to a polymorphism in a nucleotide sequence associated with a neurodegenerative disorder.

Another aspect of the invention encompasses a method for identifying a subject at risk for a neurodegenerative disorder. The method comprises determining the identity of at least one polymorphism in the subject in a nucleotide sequence selected from the group consisting of PPP3R1, GSK3β, PPP3CA, FYN, WISP1, MGEA5, CTSD, F2, MAPT, OGT and PRKCA. The polymorphism shows linkage disequilibrium and has a correlation value of greater than about 0.7 when compared to a polymorphism in a nucleotide sequence associated with a neurodegenerative disorder, wherein the presence of one allele of the polymorphism is associated with increased risk, earlier age at onset, or/and more rapid progression for the neurodegenerative disorder.

Yet another aspect of the invention encompasses a method for treating a neurodegenerative disorder in a subject. The method comprises administering to the subject an agent that increases the activity and/or the level of protein phosphatase 3.

Still another aspect of the invention encompasses a method for identifying at least one polymorphism in a subject. The polymorphism is in a nucleotide sequence selected from the group consisting of PPP3R1, GSK3β, PPP3CA, FYN, WISP1, MGEA5, CTSD, F2, MAPT, OGT and PRKCA, and shows linkage disequilibrium with a correlation value of greater than about 0.7 when compared to a polymorphism in a nucleotide sequence associated with a neurodegenerative disorder. The method comprises detecting the hybridization of a probe comprising at least one allele specific oligonucleotide whose sequence is complementary to a single nucleotide polymorphism (SNP) nucleic acid, the SNP nucleic acid being selected from the group consisting of SEQ ID NOs:1-28 to a nucleic acid sample from the subject.

A further aspect of the invention encompasses a kit for SNP genotyping a subject. The kit comprises at least one allele specific oligonucleotide that is complementary to a single nucleotide polymorphism (SNP) nucleic acid, the SNP nucleic acid being selected from the group consisting of SEQ ID NOs:1-28.

Other aspects and iterations of the invention are described more thoroughly below.

### REFERENCE TO COLOR FIGURES

The application file contains at least one photograph executed in color. Copies of this patent application publication with color photographs will be provided by the Office upon request and payment of the necessary fee.

### BRIEF DESCRIPTION OF THE FIGURES

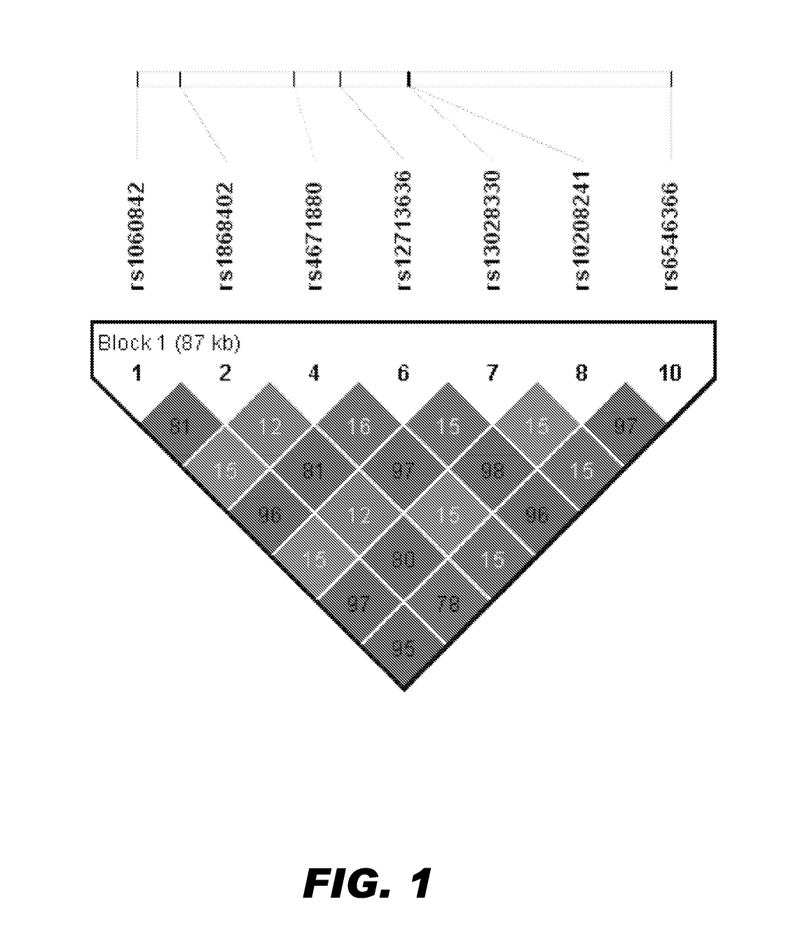

FIG. 1 depicts a linkage disequilibrium plot of the SNPs within the PPP3R1 gene.

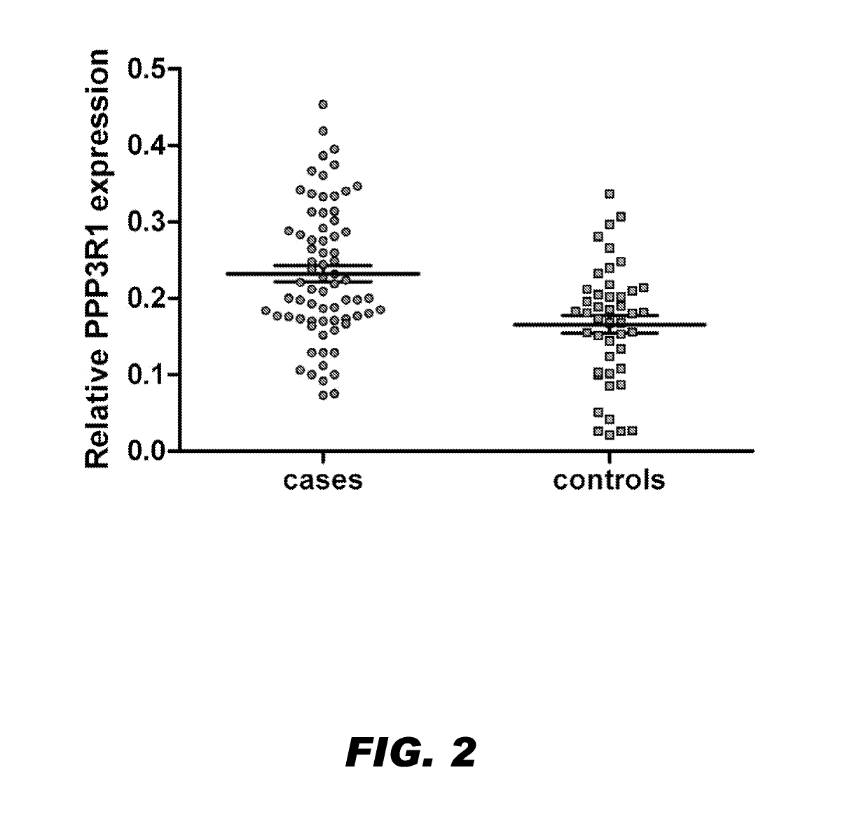

FIG. 2 illustrates the differential expression of PPP3R1. Plotted is the relative mRNA expression of PPP3R1 in brain from individuals with neuropathologically confirmed Alzheimer's disease (i.e., cases) and non-demented individuals with no Alzheimer's disease neuropathology (i.e., controls). The P-value between the two conditions was 0.0001.

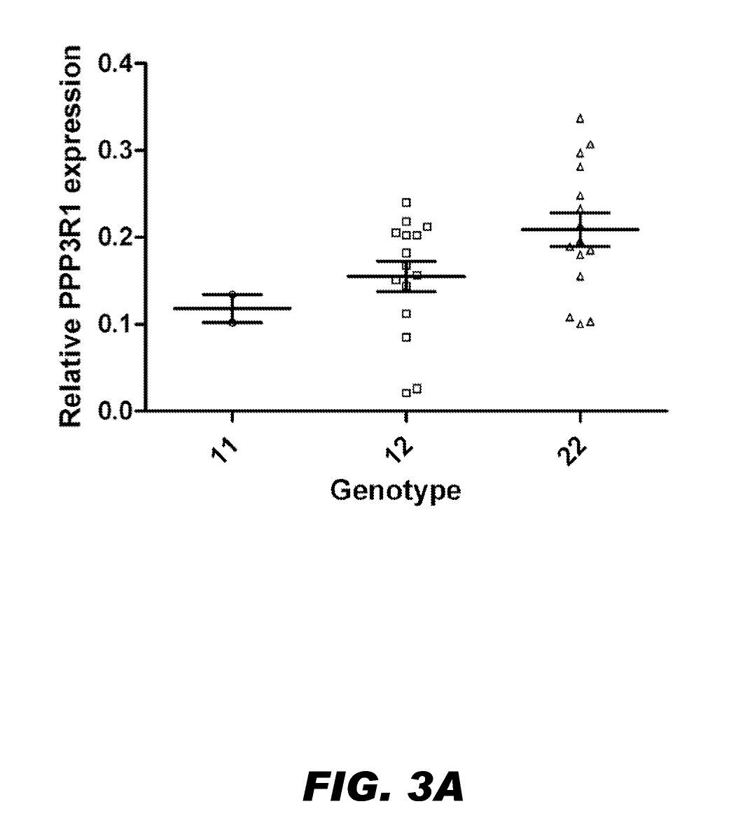

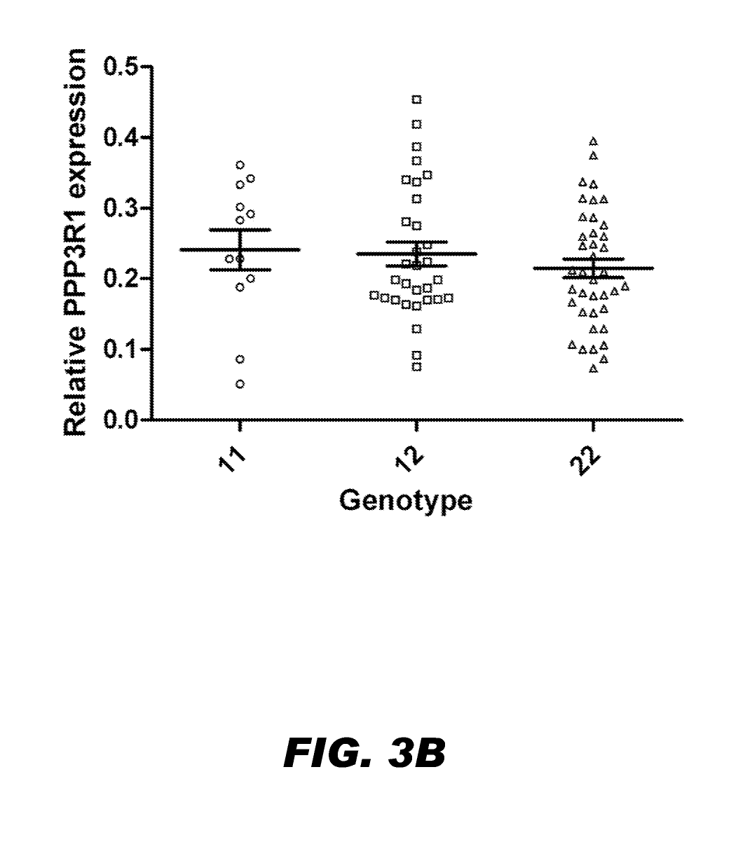

FIG. 3 depicts the association between the SNP rs1868402 and PPP3R1 mRNA expression in autopsy brain samples. Panel A presents the relative mRNA expression of PPP3R1 in the three genotypes in individuals with no dementia and no Alzheimer's disease pathology (i.e., controls). The P-value among genotypes in the controls was 0.009. Panel B presents the relative mRNA expression of PPP3R1 in individuals who had neuropathologically confirmed Alzheimer's disease (i.e., cases). The P-value among genotypes in the cases was 0.277.

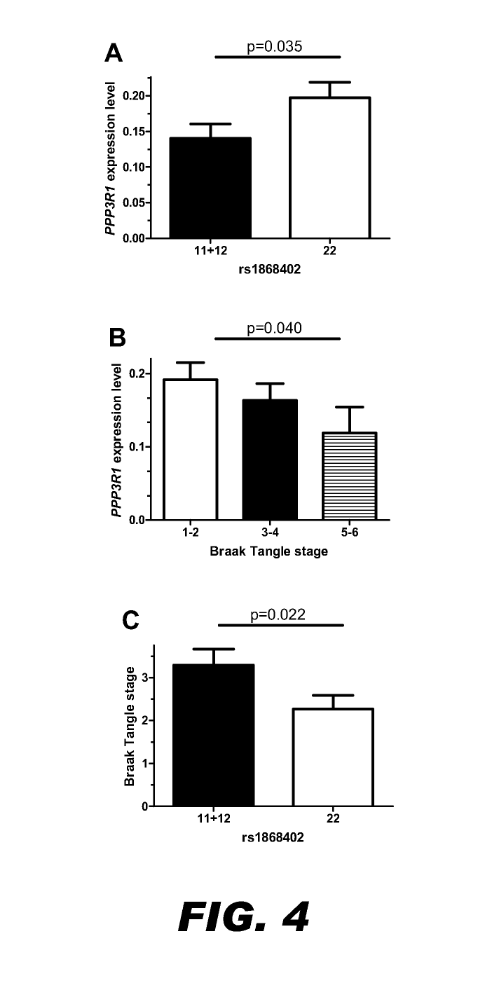

FIG. 4 depicts a series of graphs showing that Rs1868402 is associated with PPP3R1 mRNA expression and tangles counts. A: Minor allele carriers of rs1868402 have significantly lower PPP3R1 mRNA levels in non-demented individuals with AD pathology (n=22). B: PPP3R1 mRNA expression correlates with tangles counts in non-demented individuals with AD pathological changes. C: Minor allele carriers of rs1868402 have significantly higher numbers of tangles. Minor allele was coded as 1; 2 represents the major allele.

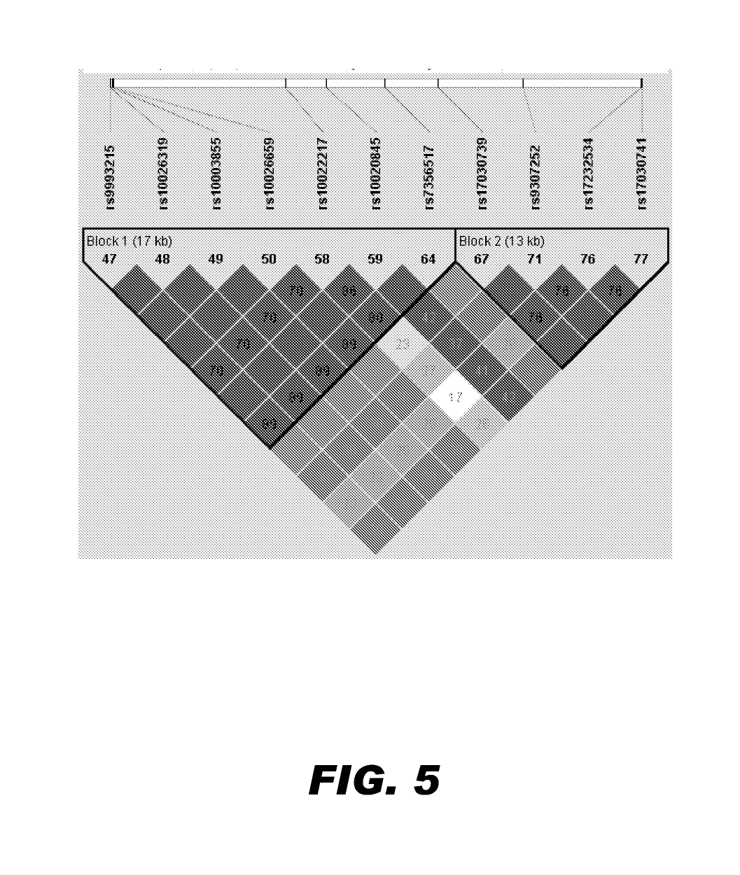

FIG. 5 illustrates the linkage disequilibrium of the imputed PPP3CA SNPs showing association with CSF ptau₁₈₁ levels in the ADRC series. Color represents D′ and numbers r².

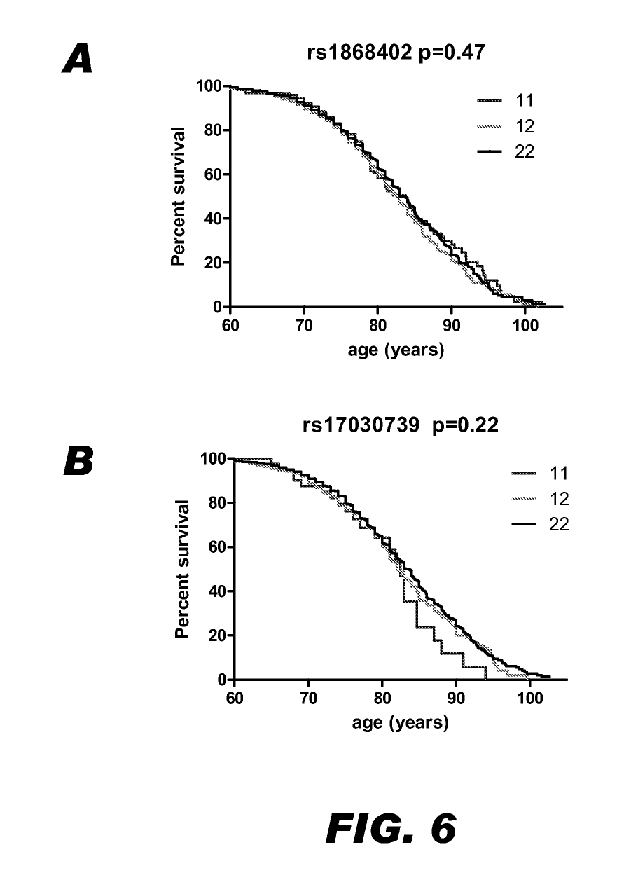

FIG. 6 depicts graphs showing survival curves comparing age at onset of LOAD between the different genotypes of rs1868402 and rs17030739.

### DETAILED DESCRIPTION OF THE INVENTION

In accordance with the present invention, it has been discovered that the level of expression of the regulatory subunit of protein phosphatase 3 (which is encoded by PPP3R1) influences the risk for neurodegenerative disorders such as Alzheimer's disease. A common polymorphism in PPP3R1 is associated with higher CSF levels of tau protein and lower mRNA expression of PPP3R1 in the brains of healthy subjects. Lower expression of PPP3R1 is predicted to increase the levels of phosphorylated tau protein in neuronal cells of the brain, thereby facilitating tangle formation and cell death. Increasing the activity of protein phosphatase 3 may block or partially reverse the formation of tau tangles. The inventors have also discovered additional polymorphisms in PPP3R1 and other nucleotide sequences that are associated with higher CSF tau levels.

Generally speaking, the present invention encompasses biomarkers, methods of using the biomarkers to assess risk for a neurodegenerative disorder, and methods of treating a neurodegenerative disorder. Also provided are oligonucleotides and kits comprising the oligonucleotides that may be used to determine the identity (or genotype) of the polymorphisms.

### (I) Biomarker for a Neurodegenerative Disorder

One aspect of the present invention encompasses a biomarker for a neurodegenerative disorder. The biomarker comprises at least one polymorphism in a nucleotide sequence selected from the group consisting of PPP3R1, GSK3β, PPP3CA, FYN, WISP1, MGEA5, CTSD, F2, MAPT, OGT and PRKCA, wherein the polymorphism shows linkage disequilibrium and has a correlation value of greater than about 0.7 when compared to a polymorphism in a nucleotide sequence associated with a neurodegenerative disorder. In general, the polymorphism of the invention is associated with the level of tau protein and/or phosphorylated tau protein in a subject.

Each of the above listed nucleotide sequences (or genes) give rise to a protein product that is associated with the phosphorylation/dephosphorylation of tau protein or the metabolism of tau protein. In particular, PPP3R1 encodes the alpha isoform of the regulatory subunit B of protein phosphatase 3 (formerly called protein phosphatase 2B); GSK3β encodes glycogen synthase kinase 3 beta; PPP3CA encodes the alpha isoform of the catalytic subunit of protein phosphatase 3; FYN encodes a tyrosine kinase related to Src; WISP1 encodes WNT1 inducible signaling pathway protein 1; MGEA5 encodes meningioma expressed antigen 5 (hyaluronidase); CTSD encodes cathepsin D; F2 encodes coagulation factor II (thrombin); MAPT encodes microtubule associated protein tau; OGT encodes O-linked N-acetylglucosamine transferase; and PRKCA encodes protein kinase C alpha.

The polymorphism may be an insertion, a deletion, or a single nucleotide polymorphism (SNP). In a preferred embodiment, the polymorphism is a SNP. The SNP may be selected from the group consisting of rs1060842, rs1868402, rs4671880, rs12713636, rs13028330, rs10208241, rs6546366, rs7431209, rs17030739, rs927010, rs7768046, rs2930000, rs2305192, rs7218425, rs1317356, rs2070852, rs7210728, rs6525488, rs9307252, rs17030741, rs9993215, rs10026319, rs10003855, rs10026659, rs10022217, rs10020845, rs7356517, rs9307252, rs17232534, rs17030741, and combinations thereof. In a preferred embodiment, the nucleotide sequence is PPP3R1 and the SNP may be selected from the group consisting of rs1060842, rs1868402, rs4671880, rs12713636, rs13028330, rs10208241, rs6546366, and combinations thereof. In another preferred embodiment, the nucleotide sequence is PPP3CA and the SNP may be rs9993215, rs10026319, rs10003855, rs10026659, rs10022217, rs10020845, rs7356517, rs17030739, rs9307252, rs17232534, rs17030741, and combinations thereof. In an exemplary embodiment, the nucleotide sequence is PPP3R1 and the SNP may be rs1868402. In another exemplary embodiment, the nucleotide sequence is PPP3CA and the SNP may be rs9307252, rs17030741, rs17030739, and combinations thereof. Table A presents the location of each of the above listed SNPs in the corresponding gene or nucleotide sequence.

TABLE A

| SNP | Gene | Location |
|---|---|---|
| rs1060842 | PPP3R1 | 3′ UTR |
| rs1868402 | PPP3R1 | intron |
| rs4671880 | PPP3R1 | intron |
| rs12713636 | PPP3R1 | intron |
| rs13028330 | PPP3R1 | intron |
| rs10208241 | PPP3R1 | intron |
| rs6546366 | PPP3R1 | flanking 5′ UTR |
| rs7431209 | GSK3β | intron |
| rs927010 | FYN | intron |
| rs7768046 | FYN | flanking 5′ UTR |
| rs2930000 | WISP1 | intron |
| rs2305192 | MGEA5 | intron |
| rs7218425 | PRKCA | intron |
| rs1317356 | CTSD | intron |
| rs2070852 | F2 | intron |
| rs7210728 | MAPT | intron |
| rs6525488 | OGT | intron |
| rs9993215 | PPP3CA | intron |
| rs10026319 | PPP3CA | intron |
| rs10003855 | PPP3CA | intron |
| rs10026659 | PPP3CA | intron |
| rs10022217 | PPP3CA | intron |
| rs10020845 | PPP3CA | intron |
| rs7356517 | PPP3CA | intron |
| rs17030739 | PPP3CA | intron |
| rs9307252 | PPP3CA | intron |
| rs17232534 | PPP3CA | intron |
| rs17030741 | PPP3CA | intron |

As detailed in the Examples, the SNPs of the invention are associated with the levels of tau protein and tau protein phosphorylated at amino acid 181 (p-tau₁₈₁) in the cerebrospinal fluid (CSF) of a subject. Tau protein is a microtubule-binding protein found predominately in neuronal cells of the central nervous system. It is well known in the art that tangles of tau protein filaments accumulate in the neuronal cells of subjects with neurodegenerative disorders. The tangles of tau protein filaments may comprise tau protein, phosphorylated tau protein, and hyperphosphorylated tau protein. The tau protein may be one of several isoforms generated by alternate splicing. The isoform may be 0N3R, 0N4R, 1N3R, 1N4R, 2N3R, or 2N4R. The phosphorylated tau protein may comprise a phosphate group on T181, S199, S202, T205, T212, S214, T217, T231, S262, S356, S393, S396, S400, S404, S409, S422, or combinations thereof.

In general, the biomarker will serve as an indicator of a neurodegenerative disorder. Typically, the neurodegenerative disorder will be a tauopathy. The term “tauopathy” refers to a group of diverse dementias and movement disorders that have as a common pathological feature the presence of intracellular accumulations of abnormal filaments of tau protein. Non-limiting examples of tauopathies include Alzheimer's disease, amyotrophic lateral sclerosis/parkinsonism-dementia complex, argyrophilic grain dementia, corticobasal degeneration, Creutzfeldt-Jakob disease, dementia pugilistica, diffuse neurofibrillary tangles with calcification, Down's syndrome, frontotemporal dementia with Parkinsonism linked to chromosome 17, Gerstmann-Sträussler-Scheinker disease, Guadeloupean parkinsonism, Hallevorden-Spatz disease, inclusion-body myositis, multiple system atrophy, Niemann-Pick disease type C, Pick's disease, prion protein cerebral amyloid angiopathy, progressive subcortical gliosis, progressive supranuclear palsy, sporadic frontotemporal dementia, subacute sclerosing panencephalitis, and tangle-predominant Alzheimer's disease. In preferred embodiments, the tauopathy may be Alzheimer's disease, corticobasal degeneration, frontotemporal dementia with Parkinsonism linked to chromosome 17, Pick's disease, progressive supranuclear palsy, sporadic frontotemporal dementia, and subacute sclerosing panencephalitis. In an exemplary embodiment, the neurodegenerative disorder may be Alzheimer's disease. The Alzheimer's disease may be early onset, rapid onset, or late onset.

As stated above, a polymorphism of the invention shows linkage disequilibrium and has a correlation value (r²) of greater than about 0.7. In one embodiment, the correlation value is about 0.7 or higher. In yet another embodiment, the correlation value is 0.9 or higher. In some embodiments, the correlation value is about 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, or higher. In each of the above embodiments, the correlation value is an r² value. Methods of calculating the correlation value as an r² value are known in the art.

The correlation value is calculated in comparison to a polymorphism in a nucleotide sequence associated with a neurodegenerative disorder. Generally speaking, a nucleotide sequence associated with a neurodegenerative disorder is a nucleotide sequence associated with tau or tau phosphorylation, or a nucleotide sequence that encodes a polypeptide associated with tau or tau phosphorylation. For instance, such a nucleic acid or polypeptide sequence may be associated with tau transcription, tau translation, tau stability, tau degredation, tau phosphorylation or tau de-phosphorylation. In one embodiment, the correlation value is calculated in comparison to a polymorphism in a nucleotide sequence associated with a gene listed in Table A. For example, the correlation value may be calculated in comparison to a polymorphism in an intron, an untranslated region (3′ or 5′), a promoter sequence, a regulatory sequence, or an exon of a gene listed in Table A.

### (II) Method for Identifying a Subject at Risk for a Neurodegenerative Disorder

Another aspect of the invention provides a method for identifying a subject at risk for a neurodegenerative disorder. The method comprises determining the identity of at least one polymorphism in the subject in a nucleotide sequence selected from the group consisting of PPP3R1, GSK3β, PPP3CA, FYN, WISP1, MGEA5, CTSD, F2, MAPT, OGT and PRKCA, wherein the polymorphism shows linkage disequilibrium and has a correlation value of greater than about 0.7 when compared to a polymorphism in a nucleotide sequence associated with a neurodegenerative disorder. The presence of one allele of the polymorphism is associated with increased risk, earlier age at onset, or/and more rapid progression for the neurodegenerative disorder.

As detailed above, the polymorphism is preferably a SNP. The SNP may be selected from the group consisting of rs1060842, rs1868402, rs4671880, rs12713636, rs13028330, rs10208241, rs6546366, rs7431209, rs17030739, rs927010, rs7768046, rs2930000, rs2305192, rs7218425, rs1317356, rs2070852, rs7210728, rs6525488, rs9307252, rs17030741, rs9993215, rs10026319, rs10003855, rs10026659, rs10022217, rs10020845, rs7356517, rs9307252, rs17232534, rs17030741, and combinations thereof. In a preferred embodiment, the nucleotide sequence is PPP3R1 and the SNP may be selected from the group consisting of rs1060842, rs1868402, rs4671880, rs12713636, rs13028330, rs10208241, rs6546366, and combinations thereof. In another preferred embodiment, the nucleotide sequence is PPP3CA and the SNP may be rs9993215, rs10026319, rs10003855, rs10026659, rs10022217, rs10020845, rs7356517, rs17030739, rs9307252, rs17232534, rs17030741, and combinations thereof. In an exemplary embodiment, the nucleotide sequence is PPP3R1 and the SNP may be rs1868402, wherein the presence of C rather than T is associated with increased risk for the neurodegenerative disorder. In another exemplary embodiment, the nucleotide sequence is PPP3CA and the SNP may be rs9307252, wherein the presence of C rather than T is associated with increased risk for the neurogegenerative disorder; rs17030741, wherein the presence of A rather than G is associated with increased risk for the neurogegenerative disorder; rs17030739, wherein the presence of A rather than G is associated with increased risk for the neurogegenerative disorder; and combinations thereof.

A SNP generally comprises two alleles: a major allele and a minor allele. The major allele is defined as the allele in a given population that has a higher allele frequency. The subject may be homozygous or heterozygous for a given SNP. As used herein, homozygous refers to a subject that has the same nucleotide on both chromosomes at a given position. Heterozygous, as used herein, refers to a subject that has a different nucleotide on each chromosome at a given position. As used herein, the phrase “determining the identity of a SNP” refers to identifying the nucleotide at the SNP position on one or both chromosomes. When the identity of the SNP nucleotide is determined on both chromosomes, it is called genotyping.

Techniques for identifying SNPs involve procedures well known in the field of molecular genetics. Many, but not all, of the methods involve amplification of nucleic acids. Ample guidance for performing amplification is provided in the art. Exemplary references include manuals such as PCR Technology: Principles and Applications for DNA Amplification (ed. H. A. Erlich, Freeman Press, NY, N.Y., 1992); PCR Protocols: A Guide to Methods and Applications (eds. Innis, et al., Academic Press, San Diego, Calif., 1990); Current Protocols in Molecular Biology (Ausubel et al., John Wiley & Sons, New York, 2003); Molecular Cloning: A Laboratory Manual (Sambrook & Russell, Cold Spring Harbor Press, Cold Spring Harbor, N.Y., 3rd Ed, 2001). General methods for detection of single nucleotide polymorphisms is disclosed in Single Nucleotide Polymorphisms: Methods and Protocols, Pui-Yan Kwok, ed., 2003, Humana Press.

Although many of the methods typically employ PCR steps, other amplification protocols may also be used. Suitable amplification methods include ligase chain reaction (see, e.g., Wu & Wallace, Genomics 4:560-569, 1988); strand displacement assay (see, e.g. Walker et al., Proc. Natl. Acad. Sci. USA 89:392-396, 1992; U.S. Pat. No. 5,455,166); and several transcription-based amplification systems, including the methods described in U.S. Pat. Nos. 5,437,990; 5,409,818; and 5,399,491; the transcription amplification system (TAS) (Kwoh et al., Proc. Natl. Acad. Sci. USA 86:1173-1177, 1989); and self-sustained sequence replication (3SR) (Guatelli et al., Proc. Natl. Acad. Sci. USA 87:1874-1878, 1990; WO 92/08800). Alternatively, methods that amplify the probe to detectable levels may be used, such as Qβ-replicase amplification (Kramer & Lizardi, Nature 339:401-402, 1989; Lomeli et al., Clin. Chem. 35:1826-1831, 1989). A review of known amplification methods is provided, for example, by Abramson and Myers in Current Opinion in Biotechnology 4:41-47, 1993. Each of the afore-mentioned references is incorporated herein in its entirety.

Oligonucleotides for amplification or other procedures may be synthesized using commercially available reagents and instruments. Methods of synthesizing oligonucleotides are well known in the art. Alternatively, oligonucleotides may be purchased through commercial sources. On some embodiments, the oligonucleotide may be detectably labeled, for example, with a fluorescent moiety, a radioactive moiety, a luminescent chelate moiety, or a biotin moiety. In some embodiments, the oligonucleotide may be detectably labeled with a fluorescent moiety attached to the 5′-end of the oligonucleotide. In some embodiments, the oligonucleotide may further comprise a quencher moiety that quenches the fluorescent moiety when the oligonucleotide is intact or unbound.

Methods suitable for detection of the polymorphism are well known in the art. Suitable assays include allele-specific real time PCR, 5′-nuclease assays, oligonucleotide ligase assays, allele specific oligonucleotide ligation, template-directed dye-terminator incorporation, molecular beacon allele-specific oligonucleotide assays, assays employing invasive cleavage with Flap nucleases, allele-specific hybridization (ASH), dynamic allele-specific hybridization, microarray based hybridization, allele-specific ligation, primer extension, single-base extension (SBE) assays, sequencing, pyrophosphate sequencing, real-time pyrophosphate sequencing, sequence length polymorphism analysis, restriction length fragment polymorphisms (RFLP), RFLP-PCR, single-stranded conformational polymorphism (SSCP), PCR-SSCP, ARMS-PCR, fragment sizing capillary electrophoresis, temperature gradient gel electrophoresis, denaturing high performance liquid chromatography, high resolution melting of the amplicon, heteroduplex analysis, and mass array systems. Analysis of amplified sequences may be performed using various technologies such as microchips, fluorescence polarization assays, and matrix-assisted laser desorption ionization (MALDI) mass spectrometry. In a preferred embodiment, the polymorphism is genotyped using the Sequenom MassArray technology (http://www.sequenom.com).

The identity of the SNP may be determined in the subject in vivo or in vitro. Typically, the SNP will be detected in vitro by identifying the nucleotide in a sample of nucleic acids obtained from the subject. To analyze SNPs, the nucleic acid sample generally comprises genomic DNA. The nucleic acid may be isolated from a biological sample using methods commonly known in the art. A skilled artisan would appreciate that the method of isolation can and will vary depending on the nucleic acid to be isolated and the biological sample used. For more information, see Ausubel et al., supra, or Sambrook & Russell, supra. Commercially available DNA or RNA extraction kits or commercially available extraction reagents may be used to isolate the nucleic acid from the biological sample.

Non-limiting examples of suitable biological samples include fluid samples, biopsy samples, skin samples, and hair samples. Fluid samples may include blood, serum, saliva, tears, and lymph. Furthermore, a lymphoblastoid cell line may be derived from the subject. Nucleic acid may be isolated from a blood sample, a saliva sample, an epithelial sample, a skin sample, a hair sample, a lymphoblastoid cell line, or other biological sample commonly used in the art. Methods of collecting a biological sample from a subject are well known in the art. In particular, methods of collecting blood samples, saliva samples, epithelial samples, and skin samples are well known in the art.

In general, the subject used in the method of the invention will be a human. Without departing from the scope of the invention, however, other mammalian subjects may be used. Suitable mammalian subjects include; companion animals, such as cats and dogs; livestock animals, such as cows, pigs, horses, sheep, and goats; zoo animals; and research animals, such as non-human primates and rodents.

### (III) Method for Treating a Neurodegenerative Disorder

Still another aspect of the invention encompasses a method for treating a neurodegenerative disorder in a subject. The method comprising administering to the subject an agent that increases the activity and/or the level of protein phosphatase 3 (calcineurin). As detailed above, the regulatory subunit of protein phosphatase 3 is encoded by the PPP3R1 nucleotide sequence. The catalytic subunit is encoded by the PPP3CA nucleotide sequence. Increased activity or increased levels of protein phosphatase 3 may lead to decreased levels of phosphorylated and hyperphosphorylated tau proteins, which in turn may lead to decreased formation of aggregates of tangled tau protein filaments.

The agent administered to the subject may directly or indirectly increase the activity of protein phosphatase 3. Since protein phosphatase 3 generally is activated in a Ca²⁺/calmodulin dependent manner, an indirectly acting agent may elevate the level of intracellular Ca²⁺. The agent may be a phospholipase C activator such as 2,4,6-trimethyl-N-(meta-3-trifluoromethyl-phenyl)-benzene-sulfonamide (m-3M3FBS), which may increase the intracellular level of inositol triphosphate (IP₃) that then releases intracellular stores of Ca²⁺. The agent may be an IP₃ agonist such as adenophostin A, adenophostin B, bombesin, or thrombin.

In another embodiment, the agent may directly activate protein phosphatase 3. For example, the agent may be a small organic molecule that interacts with a site on the regulatory subunit or catalytic subunit of protein phosphatase 3. Alternatively, the agent may be a peptide that interacts with a site on the regulatory subunit or catalytic subunit of protein phosphatase 3.

In a further embodiment, the agent may be a nucleic acid that encodes protein phosphatase 3, interacts with a nucleotide sequence encoding protein phosphatase 3, or interacts with a nucleotide sequence that encodes a protein that regulates the expression of protein phosphatase 3 such that the level of expression of protein phosphatase 3 is increased. The nucleic acid may be double stranded or single stranded. The nucleic acid may comprise DNA, RNA, or combinations thereof. The nucleic acid may mediate its effect via RNA interference (RNAi). The nucleic acid may be introduced into a cell as part of a viral delivery system. Alternatively, the nucleic acid may be introduced as a naked nucleic acid, a liposome, or protein/nucleic acid conjugate.

The agent used to treat the neurodegenerative disorder may be administered to the subject in accord with known methods. Typically, the agent will be administered orally, but other routes of administration such as parenteral or topical may also be used.

Preparations for oral administration generally contain inert excipients in addition to the active pharmaceutical ingredient. Oral preparations may be enclosed in gelatin capsules or compressed into tablets. Common excipients used in such preparations include pharmaceutically compatible fillers/diluents such as microcrystalline cellulose, hydroxypropyl methylcellulose, starch, lactose, sucrose, glucose, mannitol, sorbitol, dibasic calcium phosphate, or calcium carbonate; binding agents such as alginic acid, carboxymethylcellulose, microcrystalline cellulose, gelatin, gum tragacanth, or polyvinylpyrrolidone; disintegrating agents such as alginic acid, cellulose, starch, or polyvinylpyrrolidone; lubricants such as calcium stearate, magnesium stearate, talc, silica, or sodium stearyl fumarate; glidants such as colloidal silicon dioxide; sweetening agents such as sucrose or saccharin; flavoring agents such as peppermint, methyl salicylate, or citrus flavoring; coloring agents; and preservatives such as antioxidants (e.g., vitamin A, vitamin C, vitamin E, or retinyl palmitate), citric acid, or sodium citrate. Oral preparations may also be administered as aqueous suspensions, elixirs, or syrups. For these, the active ingredient may be combined with various sweetening or flavoring agents, coloring agents, and, if so desired, emulsifying and/or suspending agents, as well as diluents such as water, ethanol, glycerin, and combinations thereof.

For parenteral administration (including subcutaneous, intradermal, intravenous, intramuscular, and intraperitoneal), the preparation may be an aqueous or an oil-based solution. Aqueous solutions may include a sterile diluent such as water, saline solution, a pharmaceutically acceptable polyol such as glycerol, propylene glycol, or other synthetic solvents; an antibacterial and/or antifungal agent such as benzyl alcohol, methyl paraben, chlorobutanol, phenol, thimerosal, and the like; an antioxidant such as ascorbic acid or sodium bisulfite; a chelating agent such as ethylenediaminetetraacetic acid; a buffer such as acetate, citrate, or phosphate; and/or an agent for the adjustment of tonicity such as sodium chloride, dextrose, or a polyalcohol such as mannitol or sorbitol. The pH of the aqueous solution may be adjusted with acids or bases such as hydrochloric acid or sodium hydroxide. Oil-based solutions or suspensions may further comprise sesame, peanut, olive oil, or mineral oil.

For topical (e.g., transdermal or transmucosal) administration, penetrants appropriate to the barrier to be permeated are generally included in the preparation. Transmucosal administration may be accomplished through the use of nasal sprays, aerosol sprays, tablets, or suppositories, and transdermal administration may be via ointments, salves, gels, patches, or creams as generally known in the art.

The amount of agent that is administered to the subject can and will vary depending upon the type of agent, the subject, and the particular mode of administration. Those skilled in the art will appreciate that dosages may also be determined with guidance from Goodman & Goldman's The Pharmacological Basis of Therapeutics, Tenth Edition (2001), Appendix II, pp. 475-493, and the Physicians' Desk Reference.

Examples of neurodegenerative disorders are detailed above in section (I). Suitable subjects are detailed above in section (II).

### (IV) SNP Probe

A further aspect of the invention encompasses a probe that may be used to identify a SNP in a biomarker nucleotide sequence of the invention. The probe comprises at least one allele specific oligonucleotide whose sequence is complementary to a SNP nucleic acid selected from the group consisting of SEQ ID NOs:1-28, which are presented in Table B.

TABLE B

| | | SEQ |
|---|---|---|
| | | ID |
| SNP | Sequence (5′ to 3′) | NO: |
| rs1060842 | TTACAGTGGTCGGTCACAAGAAACCA[G/T] | 1 |
| | CTGAACAATTTCAGTCATTTGAAGC | |
| rs1868402 | CCCAAATGATACAGATACTACACTTA[C/T] | 2 |
| | ACATTACCATGTCAGTATTCACTGA | |
| rs4671880 | CCTACTGAAGGCCTGTGTATTATTGT[C/T] | 3 |
| | TTTCTCCTTTTGGTTTATCCACATT | |
| rs12713636 | GTAATGACTCAAATAATAGTTCTCAA[C/G] | 4 |
| | TCCAGAAGCCCACTATAGTCTACAA | |
| rs13028330 | GTGTGTGGGCTATAGTTTATTGATCC[C/T] | 5 |
| | TGAACCTAATGCATAATTACATTTA | |
| rs10208241 | AAATTATATCTAAATATGAAAAAAGT[C/T] | 6 |
| | CTAAAGCTAACATGTTTTAAGTTTA | |
| rs6546366 | TATCGGTTCTCTTTGTAATAATTAAA[A/C] | 7 |
| | TTGTCATATACATTTGTTTTTTATT | |
| rs7431209 | CTTCTATTATTAATGGTTCTACTTTG[A/G] | 8 |
| | TAACCCTTTTATTATTGTGAAGCTT | |
| rs17030739 | AAAATACATTTTCCCAGAGGTTCATT[A/G] | 9 |
| | CAATTTTTGGCCAAACTGAGTTGTT | |
| rs927010 | AAAAGGAATACAGGGAGGAGTGAAGA[C/G] | 10 |
| | GCCAGAGGACTGTCAGGTTCCATTA | |
| rs7768046 | CAATGGGGGAGACTCCCAGGGCAACA[A/G] | 11 |
| | CAATTAGGAGGAAGGGAAAGTAAAT | |
| rs2930000 | GCCCTTAGGTGTGAAGCCTTAAGAAA[C/T] | 12 |
| | GGATGCTTTGAGTCCCAGGGCTGAG | |
| rs2305192 | CTATCCTATATGAAGTGGTTTGGTGA[A/G] | 13 |
| | GGGTCTTGCTTTTATTTGAATTTTT | |
| rs7218425 | GGGAGGCATCTCAAGGATTCTCCTTC[C/T] | 14 |
| | AGGGGATGAAATGGAGCTCAAGGAA | |
| rs1317356 | CATCTCTCGGGCTCCTGGCCCAGGCT[A/G] | 15 |
| | TGTCTTGTTCCCAGCGCTGAGGGC | |
| rs2070852 | AACAGCCTCCTGTTGGGCAATTTCCT[C/G] | 16 |
| | TTCCAGAATCAACTCCACTACCCAT | |
| rs7210728 | TCTGCTTAAGATTGTTTCTAGCATAC[A/G] | 17 |
| | TTATTTCAATTTAGGCAAATGTGAC | |
| rs6525488 | AATACCAAAAAATATCAATTTTCTGT[A/G] | 18 |
| | GCATTACCAGCCATTAGGCTTAATT | |
| rs9993215 | TTTGAAAGTAATGATTTGGGGGTAAT[C/T] | 19 |
| | CCTTTATGCTTGGGGTGCCAAGGCA | |
| rs10026319 | GCATTTCTACAAAGGAATTACTAGTG[C/T] | 20 |
| | CAAACGCTTTTTGCTAGGTCAAACA | |
| rs10003855 | TTCCTCAAGGACCAGTAAGATACAAC[A/G] | 21 |
| | TTAGTCACACTAATTCCCAACTTTG | |
| rs10026659 | ACTCTGGAACCTGTGGTATTAGATAC[A/G] | 22 |
| | AAGAATTATCTAATTCAAGGCAGAC | |
| rs10022217 | CGTTTGAGTGAAACAGGATGCAATTT[A/T] | 23 |
| | CAAGCAAGGGTAAGTCTCATCCAAT | |
| rs10020845 | TCCTGAAGCAGGACCCATGCAACTCA[C/T] | 24 |
| | AATGTTCTATGGCAGCATTTGAAGA | |
| rs7356517 | CATAACACTCAGGTAATTTTAAAGTG[A/G] | 25 |
| | TAACAAATGACTCTTCATTTCAAAA | |
| rs9307252 | AATGCAATTCCTAGATGCAGTATATA[C/T] | 26 |
| | AAGCATTTTTGCCTAGACTAAGTAA | |
| rs17232534 | AGGTCTTGAATTCACAGTGGGAAAGA[C/G] | 27 |
| | GAAAGGCAGCATGGTTTAGATGCTT | |
| rs17030741 | GTGATATTTATAAAGATGTGATGACT[A/G] | 28 |
| | GTGGTGATTTCAATACACGAGGAAA | |

In one embodiment, the probe may comprise one allele specific oligonucleotide that is complementary to the major allele of the SNP. In another embodiment, the probe may comprise one allele specific oligonucleotide that is complementary to the minor allele of the SNP. In still another embodiment, the probe may comprise a first allele specific oligonucleotide that is complementary to the major allele of the SNP and a second allele specific oligonucleotide that is complementary to the minor allele of the SNP.

The length of the allele specific oligonucleotide can and will vary. Typically, the allele specific oligonucleotide will be complementary to one allele of the SNP and from about 7 to about 15 contiguous nucleotides on each side of the SNP. In one embodiment, the allele specific oligonucleotide may comprise at least about 15 nucleotides having complementarity with the SNP nucleic acid. In another embodiment, the allele specific oligonucleotide may comprise about 17 nucleotides, about 19 nucleotides, about 21 nucleotides, about 23 nucleotides, about 25 nucleotides, about 27 nucleotides, about 29 nucleotides, or about 31 nucleotides having complementarity with the SNP nucleic acid.

Generally, the allele specific oligonucleotide will be completely complementary to the SNP and the nucleotides that flank the SNP. Stated another way, there will be 100% complementarity between the allele specific oligonucleotide and the SNP nucleic acid. Conditions under which only completely complementary nucleic acid strands will hybridize are referred to as “stringent” hybridization conditions. Stringent conditions, under which an oligonucleotide will hybridize only to the exactly complementary target sequence, are well known in the art (see, e.g. Ausubel et al., supra or Sambrook & Russell, supra). Stringent conditions are sequence dependent and will be different in different circumstances. Generally, stringent conditions are selected to be about 5° C. lower than the thermal melting point (Tm) for the specific sequence at a defined ionic strength and pH. The Tm is the temperature (under defined ionic strength and pH) at which 50% of the base pairs have dissociated. Those skilled in the art of nucleic acid technology are able to determine duplex stability empirically considering a number of variables including, for example, the length and base pair concentration of the oligonucleotides, and ionic strength.

In a further embodiment, the allele specific oligonucleotide may further comprise additional nucleotides that have no complementarity to the SNP nucleic acid. The additional non-complementary nucleotides may provide means for detection (e.g., they may base-pair to form a hairpin molecular beacon-like structure), for amplification, or for endonuclease digestion. The length of the additional nucleotides may range from about 10 to about 100 nucleotides, or more preferably from about 15 to about 40 nucleotides.

In another embodiment, the allele specific oligonucleotide may further comprise at least one moiety such as a fluorophore, a quencher, a luminescent chelate, a biotin molecule, or a radioisotope. For example, the allele specific oligonucleotide may comprise a fluorophore. Alternatively, the allele specific oligonucleotide may comprise a fluorophore and a quencher. Suitable fluorophores, quenchers, and luminescent chelates are well known in the art.

In still another embodiment, the allele specific oligonucleotide may be conjugated to a solid support. Non-limiting examples of suitable solid supports include silica, alumina, titania, carbondium, zirconia, activated charcoal, zeolite, ceramics, activated carbon, porous metal support, agarose, cellulose, nitrocellulose, methyl cellulose, polyacrylic, polyacrylamide, polyacrylonitrile, polyamide, polyether, polyester, polyethylene, polystyrene, polysulfone, polyvinyl chloride, polyvinylidene, methacrylate copolymer, and polystyrene-vinyl chloride copolymer. The solid support may be a variety of sizes and forms depending upon the embodiment of the invention. For example, the solid support may be a microarray, beads, microbeads, nanobeads, particles, nanoparticles, resins, fibers, nanofibers, nanotubes, gels, sol-gels, areogels, membranes, or a solid surface coated with a solid support. In a preferred embodiment, the solid support may be a microarray or a bead. The allele specific oligonucleotide may be conjugated to the solid support via covalent or non-covalent means.

The allele specific oligonucleotide may be synthesized by techniques well known to those with skill in the art.

A SNP probe may be used to detect a SNP in a sample from a subject. For instance, a SNP probe may be used in a method of detecting a SNP detailed in section (II) above.

### (V) Kits

Yet another aspect of the present invention provides a kit for SNP genotyping a subject. The kit comprises at least one allele specific oligonucleotide that is complementary to a single nucleotide polymorphism (SNP) nucleic acid, the SNP nucleic acid being selected from the group consisting of SEQ ID NOs:1-28. The allele specific oligonucleotides are detailed above in section (IV). The SNPs are detailed above in section (I). Suitable subjects are described above in section (II).

### DEFINITIONS

To facilitate understanding of the invention several terms are defined below.

The term “allele,” as used herein, refers to one of two or more different nucleotides that occur at a specific locus.

The term “allele specific oligonucleotide,” as used herein, refers to an oligonucleotide that is complementary to one allele of a SNP. Typically, the allele specific oligonucleotide is complementary to the central region of a SNP nucleic acid. The “central region of a SNP nucleic acid” refers to the SNP and its flanking nucleotides, i.e., about 7 to about 15 contiguous nucleotides on each side of the SNP.

As used herein, the term “complementary” refers to the natural association of two single-stranded nucleic acids by base pairing via hydrogen bonds (i.e., 5′-A G T-3′ pairs with the complimentary sequence 3′-T C A-5′). As used herein, the complementarity between two nucleic acids is complete or perfect, i.e., there are no mismatches in the region of interest (i.e., the central region of a SNP nucleic acid).

The term “linkage disequilibrium” or “LD” as used herein, refers to alleles at different loci that are not associated at random, that is, not associated in proportion to their frequencies. If the alleles are in positive linkage disequilibrium, then the alleles occur together more often than expected, assuming statistical independence. Conversely, if the alleles are in negative linkage disequilibrium, then the alleles occur together less often than expected, assuming statistical independence.

A “locus” is a chromosomal location or position. A gene locus is a specific chromosome location in the genome of a species where a specific gene can be found. A SNP locus refers to the specific nucleotide position that is polymorphic.

The term “oligonucleotide,” as used herein, refers to a single-stranded molecule comprising two or more nucleotides. The nucleotides may be standard nucleotides (i.e., adenosine, guanosine, cytidine, thymidine, and uridine) or nucleotide analogs. A nucleotide analog refers to a nucleotide having a modified purine or pyrimidine base or a modified ribose moiety. A nucleotide analog may be a naturally occurring nucleotide (e.g., inosine) or a non-naturally occurring nucleotide. Non-limiting examples of modifications on the sugar or base moieties of a nucleotide include the addition (or removal) of acetyl groups, amino groups, carboxyl groups, carboxymethyl groups, hydroxyl groups, methyl groups, phosphoryl groups, and thiol groups, as well as the substitution of the carbon and nitrogen atoms of the bases with other atoms (e.g., 7-deaza purines). Nucleotide analogs also include dideoxy nucleotides, 2′-O-methyl nucleotides, locked nucleic acids (LNA), peptide nucleic acids (PNA), and morpholinos. The nucleotides may be linked by phosphodiester, phosphothioate, phosphoramidite, or phosphorodiamidate bonds.

A “polymorphism” is a locus that is variable; that is, the nucleotide sequence at a polymorphic locus has more than one version or allele within a population. An example of a polymorphism is a single nucleotide polymorphism (SNP), which is a polymorphism at a single nucleotide position in a genome (i.e., the nucleotide at the position varies between individuals or populations). Nucleotide polymorphisms may occur at any region of a gene, that is, in the promoter region, an intron, or an exon. In some instances, the polymorphism results in a change in the protein sequence. The change in protein sequence may affect protein function or may not.

As used herein, the phrase “risk” may refer to one or more of the following: an increased risk for developing a neurodegenerative disorder, an increased risk for an earlier age at onset of a neurodegenerative disorder, and an increased risk for rapid progression of a neurodegenerative disorder.

“SNP” refers to a single nucleotide polymorphism.

As used herein, the term “SNP nucleic acid” refers to the nucleotide sequence of the region surrounding a SNP, as listed in the public database, dbSNP (http://www.ncbi.nlm.nih.gov/SNP/).

The term “treating,” as used herein, refers to alleviating, reversing, inhibiting the progress of, and/or preventing a neurodegenerative disorder, and in particular, a neurodegenerative disorder comprising the abnormal accumulation of tau proteins. The term “treatment”, as used herein, unless otherwise indicated, refers to the act of treating as “treating” is defined immediately above. In particular, the treatment may prevent, slow the progression, reverse, or partially reverse the formation of tau deposits in neuronal cells.

The following examples are included to demonstrate preferred embodiments of the invention. It should be appreciated by those of skill in the art that the techniques disclosed in the examples that follow represent techniques discovered by the inventors to function well in the practice of the invention. Those of skill in the art should, however, in light of the present disclosure, appreciate that many changes can be made in the specific embodiments that are disclosed and still obtain a like or similar result without departing from the spirit and scope of the invention, therefore all matter set forth or shown in the accompanying drawings is to be interpreted as illustrative and not in a limiting sense.

### EXAMPLES

The following examples illustrate various iterations of the invention.

### Example 1

### Identification of SNPs Associated with CSF Tau Levels

The presence of deposits of abnormally hyperphosphorylated microtubule-associated protein tau protein (MAPT) is a pathological hallmark in Alzheimer's disease (AD). The tau protein can be detected in cerebrospinal fluid (CSF) and may be a useful biomarker to predict cognitive decline in older adults because CSF tau levels are increased in neurodegenerative diseases, including AD. Phospho-tau levels correlate with the presence of tangles in the brain. Genetic epidemiology demonstrates a strong genetic component for late onset AD (LOAD). However, only APOE has been convincingly shown to influence risk for LOAD. Quantitative traits such as CSF tau levels likely are directly related to gene function and hence offer more power than qualitative case-control tests in identifying additional genetic influences for AD.

The objective of the following study was to identify SNPs in LOAD candidate genes or genes that are associated with CSF tau and phosphorylated tau₁₈₁ (p-tau₁₈₁) levels.

CSF tau and p-tau₁₈₁ were measured using standard techniques in 313 individuals; 72% were non-demented (Clinical Dementia Rating, CDR 0), 20% were very mildly demented (CDR 0.5), and 8% were mildly demented (CDR 1). CSF beta-amyloid protein (Aβ42) levels were also measured. Tagging SNPs (r²>0.8) were selected using HapMap data. SNPs with functional annotations were also included. The SNPs were genotyped using the Illumina Golden Gate and Sequenom genotyping technology. Analysis of the covariance (ANCOVA) was used to test for association between genotypes and CSF tau and p-tau₁₈₁ levels. Stepwise discriminant analyses were used to detect the significant covariates. Status, CDR, age, gender and APOE genotype were the potential covariates included in the analyses. ANCOVA model: tau/p-tau₁₈₁=CDR+APOE+age+SNP.

CSF tau and p-tau₁₈₁ levels exhibited more than ten fold variation between individuals. The CDR stage was strongly associated with both tau and p-tau₁₈₁ levels. Age showed association with CSF tau and p-tau₁₈₁ levels after correcting for CDR. APOE genotype was also associated with CSF tau/p-tau₁₈₁ levels after correcting for CDR. Since the distribution of tau and p-tau₁₈₁ levels was highly skewed, log-log transformation was used to generate a normally distributed trait.

Several SNPs were identified in genes that showed association with CSF tau/p-tau₁₈₁ levels after multiple test corrections (see Table 1). In general, the genes comprising the SNPs are related to tau dephosphorylation and beta-amyloid protein (Aβ42) metabolism. For example, the PPP3R1 gene, which codes for the regulatory subunit of the protein phosphatase 3, comprised seven SNPs. One SNP in particular (i.e., rs1868402) showed significant association with CSF tau levels.

TABLE 1
SNPs Associated with CSF Tau Levels.

| | | P-value | P-value |
|---|---|---|---|
| SNP | Gene | Tau | p-Tau₁₈₁ |
| rs1060842 | PPP3R1 | 0.0002 | 0.0036 |
| rs1868402 | PPP3R1 | 2.6 × 10⁻⁰⁵ | 0.0006 |
| rs4671880 | PPP3R1 | 0.0005 | 0.0043 |
| rs12713636 | PPP3R1 | 0.0002 | 0.0031 |
| rs13028330 | PPP3R1 | 0.0004 | 0.0031 |
| rs10208241 | PPP3R1 | 0.0002 | 0.0023 |
| rs6546366 | PPP3R1 | 0.0002 | 0.0036 |
| rs1800587 | IL1A | 0.0039 | 0.0466 |
| rs7431209 | GSK3β | 0.0077 | 0.0048 |
| rs3755557 | GSK3β | 0.0020 | 0.0039 |
| rs17030739 | PPP3CA | 0.0062 | 0.0014 |
| rs927010 | FYN | 0.0005 | 0.0005 |
| rs7768046 | FYN | 0.0007 | 0.0040 |
| rs2930000 | WISP1 | 0.0010 | 0.0183 |
| rs1800682 | FAS | 0.0085 | 0.0645 |
| rs2305192 | MGEA5 | 0.0007 | 0.0081 |
| rs7218425 | PRKCA | 0.0046 | 0.0046 |

As shown in Table 2, the association between rs1868402 and CSF tau/p-tau₁₈₁ levels was most significant at CSF Aβ42 levels of greater than 500 pg/mL.

TABLE 2
Association of rs1868402 and CSF Aβ42 Levels

| | | P-values |
|---|---|---|
| Stratum | n | Tau |
| Total sample | 342 | 2.6 × 10⁻⁰⁵ |
| CSF Aβ42 >500 pg/ml | 161 | 0.0007 |
| CSF Aβ42 <500 pg/ml | 181 | 0.22 |

FIG. 1 presents a linkage disequilibrium plot for the seven SNPs identified in the PPP3R1 gene. All of the SNPs within this gene captured the same genetic variation.

These data highlight the strength of the endophenotype-based approach and may lead to the identification of novel genes/SNPs that influence risk or onset for LOAD by modulating CSF tau levels.

### Example 2

### Differential Expression of PPP3R1 and Genotype Analysis

To more closely examine the association between PPP3R1 and LOAD, the expression of PPP3R1 was measured in autopsy brain samples of 82 individuals who had neuropathologically confirmed AD (i.e., cases) and 37 non-demented individuals with minimal AD neuropathology (i.e., controls). Expression was analyzed by reverse transcriptase, real-time PCR following standard procedures (GAPDH was used as a reference gene).

FIG. 2 presents the relative mRNA expression of PPP3R1 (i.e., relative to GAPDH) in controls and AD cases. The mRNA expression of PPP3R1 was higher in the AD cases. The P-value between the two conditions was 0.0001.

Next, individuals were genotyped for rs1868402. Those homozygous for the minor allele (C) are designated “11”; heterozygotes are designated “12”; and those homozygous for the major allele (T) are designated “22.” The relative level of expression of PPP3R1 for each genotype in the controls and AD cases are presented in FIGS. 3A and 3B, respectively. The level of mRNA expression of PPP3R1 was equally high in all genotypes for the cases, but controls with the minor allele had the lowest relative level of PPP3R1 expression. Table 3 summarizes these analyses.

TABLE 3
Genotype Analysis.

| | | PPP3R1 | P-value | P-value |
|---|---|---|---|---|
| | Genotype | Expression | Genotype | Condition |
| Cases | 11 | 0.241 | 0.277 | 0.0001 |
| | 12 | 0.235 | | |
| | 22 | 0.214 | | |
| Controls | 11 | 0.118 | 0.009 | |
| | 12 | 0.150 | | |
| | 22 | 0.210 | | |

These data reveal that PPP3R1 was differentially expressed in individuals with and without Alzheimer's disease, suggesting that protein phosphatase 3 may play a role in Alzheimer's disease and other neurodegenerative disorders. Furthermore, in controls, rs1868402 was associated with higher CSF tau levels and lower PPP3R1 mRNA levels, indicating that this SNP could be a genetic risk factor for Alzheimer's disease.

### Materials and Methods for Examples 3-5

### Subjects and Endophenotypes

The cerebrospinal fluid (CSF) discovery series included 353 individuals enrolled in longitudinal studies at the ADRC. CSF was collected by lumbar puncture after fasting as described previously (9). Age at lumbar puncture in these samples ranges from 37 to 94 years. Approximately 72% of these individuals were non-demented (Clinical Dementia Rating, CDR 0 (21)), 18% were very mildly demented (CDR 0.5), and 8% were mildly demented (CDR 1). Thirty nine percent were male and 40% carry at least one APOE ε4 allele (Table 4). CSF collection, processing, and tau and ptau₁₈₁ measurements were performed as described previously (9). A description of the CSF levels can been found in Table 5. The CSF replication series consisted of 266 individuals (40% CDR 0 and 60% CDR >0) from the ADNI dataset. Demographic data are shown in Table 4. The determinations of the CSF tau and ptau₁₈₁ levels in the ADNI samples were measured on the Luminex platform by Drs Leslie Shaw and John Trojanowski of the ADNI Biomarker Core at the University of Pennsylvania School of Medicine. (22). While there are differences in the absolute levels of the biomarker measurements (Table 5) that likely reflect differences in the methods used for quantification (regular ELISA vs Luminex), ascertainment (more AD cases), and/or in handling of the CSF after collection, CSF tau and ptau₁₈₁ levels in the ADNI and ADRC samples show similar characteristics. CSF levels have an approximately between 10-17 fold difference between the minimum and maximum, (Table 5) are normally distributed after log-log transformation, and have similar covariates in both datasets.

TABLE 4
Summary of sample characteristics

| | | | Age (yrs) | Male | APOE ε 4+ | |
|---|---|---|---|---|---|---|
| Sample | | n | Mean ± SD (range) | (%) | (%) | CDR |
| ADRC | CSF | 353 | 68 ± 11 (37-94) | 39 | 40 | 0 = 72%: >0.5 = 18% |
| ADNI | CSF | 266 | 75 ± 6 (56-91) | 56 | 47 | 0 = 40%: >0.5 = 60% |
| Expression | cases | 81 | 86 ± 7 (72-102) | 45 | 41 | >0.5 = 100% |
| | controls | 39 | 85 ± 9 (64-107) | 41 | 23 | 0 = 100% |
| WU | cases | 340 | 83 ± 7 (69-101) | 35 | 56 | >0.5 = 100% |
| | control | 281 | 78 ± 8 (60-102) | 39 | 21 | 0 = 100% |
| ADNI | cases | 247 | 71 ± 8 (52-91) | 55 | 65 | >0.5 = 100% |
| | control | 229 | 77 ± 5 (61-92) | 53 | 26 | 0 = 100% |
| Carfiff | cases | 666 | 76 ± 7 (60-97) | 27 | 61 | >0.5 = 100% |
| | control | 812 | 76 ± 6 (61-97) | 37 | 23 | 0 = 100% |

Sample size (n), age, percentage of males, percentage of APOE ε 4 allele carriers, and clinical dementia rating (CDR) for each sample.

The expression studies were carried out using cDNA obtained from the parietal lobe of 82 AD cases and 39 non-demented individuals (CDR=0) obtained through the WU-ADRC neuropathology Core (Table 4). AD changes were measured using Braak and Braak stage (23). All AD cases had a Braak and Braak score of 5 or 6. Among the non-demented individuals 24 brains had a Braak and Braak staging ranging from 1-4 indicating the presence of some tangle pathology.

Risk for disease and age at onset analyses were analyzed in a total of 1253 late-onset AD (LOAD) cases and 1322 age-gender matched non-demented controls (Table 4). These samples were ascertained at ADRC (312 cases and 262 controls), MRC genetic resource for late-onset AD (UK) (MRC Sample (24)) (666 cases and 804 controls) and ADNI (224 cases controls and 220 controls) (Table 4). The diagnosis of AD was based on international criteria (25). All individuals were Caucasian and written consent was obtained from all participants.

TABLE 5
Summary of Biomarker Characteristics.

| | ADRC | ADNI |
|---|---|---|
| Aβ42 | 564 ± 244 (175-1295) | 170 ± 56 (53-300) |
| Tau | 376 ± 241 (88-1358) | 98 ± 56 (28-495) |
| Ptau181 | 63 ± 32 (24-241) | 18 ± 8 (8-115) |

CSF Aβ42, Aβ40, tau and ptau181 levels for the Washington University Alzheimer's Disease Reseach Center (ADRC)(and Alzheimer's Disease Neuroimaging Initiative (ADNI) sample. For each phenotype the mean in pg/ml with the standard deviation and range is shown.

### SNP Selection and Genotyping

We selected single nucleotide polymorphisms (SNPs) located in genes implicated in tau phosphorylation/dephosphorylation and other related posttranslational modifications. Based on bibliographic data, we selected SNPs in the most relevant tau kinases, including glycogen synthase kinase 3 beta (GSK3β), cyclin-dependent kinase 5 (CDK5), and mitogen-activated protein kinases (MAPKs); tau phosphatases, protein phosphatase 2A (PP2A) and protein phosphatase 2B (PP2B) (26), and the O-linked N-acetylglucosamine (GlcNAc) transferase (OGT) and meningioma expressed antigen 5 (hyaluronidase) (MGEA5). OGT and MGEA5 code for enzymes implicated in tau O-glcNAcylation, a normal tau posttranslational modification that downregulates tau phosphorylation; alteration of this process could result in abnormal tau phosphorylation (27). Other genes implicated directly or indirectly in tau phosphorylation or degradation were also included in this study (Table 6). Tagging SNPs (r²>0.8), based on CEU-HapMap data, were selected for each of these genes. We used Pupasuite software to select potentially functional variants in the selected genes and neighboring regions. SNPs were genotyped using the Illumina Golden Gate, Sequenom and/or Taqman genotyping technology. Only SNPs with a genotyping call rate higher than 95% and SNPs in Hardy-Weinberg equilibrium were used in the analyses. A total of 384 SNPs were selected and 355 passed quality control. Genotypes for untyped SNPs in the protein phosphatase 3 (formerly 2B), regulatory subunit B, alpha isoform (PPP3R1) and phosphatase 3, catalytic subunit, alpha isoform (PPP3CA) were imputed based on the CEU-HAPMAP population genotypes using the MACH software package (http://www.sph.umich.edu/csg/abecasis/). Only genotypes with quality scores greater than 0.90 were included in the analyses.

TABLE 6
Candidate genes number of SNPs selected and genotyped

| | Official | Common | | Chromo- | Size | | Evol. | Pot. | Total | Pass |
|---|---|---|---|---|---|---|---|---|---|---|
| | Name | Alias | Activity | some | Kb | tSNP^{A} | Cons.^{B} | Funct.^{B} | Selected | QC^{C} |
| 1 | MARK1 | — | Phosphorylation | 1q41 | 135.7 | 7 | 4 | 1 | 12 | 8 |
| 2 | PPP3R1 | CNB | Phosphorylation | 2p15 | 73.65 | 8 | 2 | 1 | 11 | 10 |
| 3 | GSK3β | — | Phosphorylation | 3q13 | 267 | 5 | 2 | 1 | 8 | 4 |
| 4 | PPP3CA | PP2B | Desphosphorylation | 4q21 | 323.79 | 40 | 3 | 0 | 43 | 40 |
| 5 | CAST | Calpastatin | Degradation | 5q15 | 112.4 | 29 | 4 | 3 | 36 | 36 |
| 6 | PPP2CA | PP2A | Desphosphorylation | 5q31 | 28.8 | 2 | 3 | 1 | 6 | 5 |
| 7 | HSPA4 | HSP70 | Other | 5q31.1 | 53.05 | 4 | 1 | 1 | 6 | 3 |
| 8 | CSNK1A1 | CK1 | Phosphorylation | 5q32 | 56.16 | 10 | 1 | 1 | 12 | 11 |
| 9 | CAMK2A | CAMKA | Phosphorylation | 5q33 | 70.28 | 25 | 5 | 3 | 33 | 30 |
| 10 | HSPA1A | HSP70-1A | Other | 6p21 | 2.38 | 2 | 1 | 0 | 3 | 2 |
| 11 | FYN | — | Phosphorylation | 6q21 | 212.1 | 38 | 1 | 4 | 43 | 43 |
| 12 | WISP3 | — | Phosphorylation | 6q21 | 15.6 | 6 | 1 | 0 | 7 | 7 |
| 13 | PPP1R3A | PP1 | Desphosphorylation | 7q31 | 44.68 | 4 | 0 | 0 | 4 | 4 |
| 14 | CDK5 | — | Phosphorylation | 7q36 | 4.1 | 5 | 0 | 1 | 6 | 5 |
| 15 | PPP2R2A | PP2A | Desphosphorylation | 8p21.2 | 79.61 | 12 | 3 | 1 | 16 | 16 |
| 16 | WISP1 | — | Phosphorylation | 8q24 | 38.3 | 21 | 1 | 2 | 23 | 23 |
| 17 | MGEA5 | OGA | O-glcNAcylation | 10q24 | 33.97 | 4 | 1 | 1 | 6 | 5 |
| 18 | F2 | PT | Degradation | 11p11 | 20.3 | 2 | 2 | 1 | 2 | 2 |
| 19 | CTSD | — | Degradation | 11p15 | 11.24 | 3 | 1 | 1 | 3 | 3 |
| 20 | MARK2 | PAR1 | Phosphorylation | 11q12 | 70.15 | 7 | 5 | 4 | 7 | 7 |
| 21 | CAPN1 | — | Degradation | 11q13 | 30.13 | 8 | 2 | 2 | 8 | 7 |
| 22 | TTBK2 | — | Phosphorylation | 15q15 | 176.5 | 5 | 1 | 1 | 5 | 4 |
| 23 | MAPK3 | ERK1 | Phosphorylation | 16p11 | 9.2 | 4 | 2 | 2 | 4 | 2 |
| 24 | CDK5R1 | P35 | Phosphorylation | 17q11.2 | 4.17 | 6 | 2 | 1 | 6 | 6 |
| 25 | MAPT | Tau | — | 17q21 | 133.9 | 15 | 3 | 0 | 15 | 15 |
| 26 | PRKCA | PKCA | Phosphorylation | 17q22 | 507.9 | 8 | 2 | 1 | 9 | 9 |
| 27 | CSNK1D | HCKID | Phosphorylation | 17q25 | 29.33 | 4 | 1 | 0 | 4 | 4 |
| 28 | PRKACA | PKA | Phosphorylation | 19p13 | 26.05 | 4 | 1 | 1 | 4 | 3 |
| 29 | PIN1 | — | Other | 19p13 | 14.36 | 4 | 2 | 1 | 8 | 8 |
| 30 | MARK4 | — | Phosphorylation | 19q13.3 | 53.7 | 6 | 1 | 1 | 6 | 6 |
| 31 | CSNK2A1 | CKII | Phosphorylation | 20p13 | 61.15 | 11 | 2 | 1 | 11 | 11 |
| 32 | WISP2 | — | Phosphorylation | 20q12 | 12.6 | 3 | 1 | 3 | 3 | 3 |
| 33 | MAPK1 | ERK2 | Phosphorylation | 22q11 | 108 | 8 | 3 | 1 | 8 | 8 |
| 34 | OGT | — | O-glcNAcylation | Xq13 | 42.81 | 3 | 3 | 2 | 6 | 5 |
| | Total | | | | | 323 | 67 | 44 | 384 | 355 |

The official and the most common alias of the gene, activity related to tau, chromosomal position, gene size in Kb are showed.
^{A}Tag SNP. SNP that capture the 80% of the diversity of the gene
^{B}Only validated SNP with a minor allele frequency >0.1
^{C}NumberSNP passed quality controls

### Expression

Total RNA was extracted from the parietal lobe of 82 AD cases and 39 non-demented individuals, using the RNeasy mini kit (Qiagen) following the manufacturer's protocol. cDNAs were prepared from the total RNA, using the High-Capacity cDNA Archive kit (ABI). Gene expression level was analyzed by real-time PCR, using an ABI-7500 real-time PCR system. Real-time PCR assays were used to quantify PPP3R1 and PPP3CA cDNA levels. Sybr-green primers for PPP3R1 and GAPDH were designed over exon-exon boundaries, using Primer Express software, Version 3 (ABI) (sequences available on request). Taqman assays for PPP3CA (Hs00174223_m1) and GAPDH (sequences available on request) were also used to quantify the gene expression levels. Each real-time PCR run included within-plate duplicates and each experiment was performed, at least twice for each sample. Real-time data were analyzed by using the comparative Ct method. The Ct values of each sample were normalized with the Ct value for the housekeeping gene, GADPH, and were corrected for the PCR efficiency of each assay (28), although the efficiency of all reactions was close to 100%. Only samples with a standard error of <0.15% were analyzed.

### Statistical Analyses

CSF tau and ptau₁₈₁ were log-log transformed to approximate a normal distribution. Analysis of the covariance (ANCOVA) was used to test for association between genotypes and CSF levels. Stepwise discriminant analysis identified CDR (clinical dementia rating), age, and APOE genotype as important covariates in the ADRC series and, CDR and APOE genotype in the ADNI series. These covariates were included in the respective ANCOVA analysis. Each SNP was tested by using an additive model with minor allele homozygotes being coded as 0, heterozygotes being coded as 1, and major allele homozygotes being coded as 2. In cases where the additive model was significant at p<0.05, the dominant and recessive models were tested to determine whether they were a better fit.

Because the CSF tau and p-tau₁₈₁ levels in the ADRC and ADNI samples were measured using different platforms (Innotest plate ELISA vs AlzBia3 bead-based ELISA, respectively) we were not able to combine the raw data, rather we combined the residual values of the CSF tau and ptau₁₈₁ obtained after correcting for the covariates. False discovery rate (FDR, filter 0.1) was used for multiple test correction. Because each gene was selected based on its role in tau metabolism and/or its possible effects on CSF tau and ptau₁₈₁ levels, multiple test corrections were calculated for each gene region separately.

Association between cDNA levels, tau pathology (Braak tangle stage) and genotypes were carried out with ANCOVA tests. Stepwise discriminant analysis was used to determine the significant covariates (age, gender, postmortem interval, APOE genotype, and CDR) in each case. One-tailed P-values were calculated, because a priori predictions were made based on associations with CSF tau/p-tau₁₈₁ levels.

Allelic and genotypic association with risk for AD was tested using Fisher's exact test. Association with age at onset (AAO) was carried out using the Kaplan-Meier method and tested for significant differences, using a log-rank test. One-tailed P-values were calculated, because a priori predictions were made based on associations with CSF tau/p-tau₁₈₁ levels.

### ADNI Material and Methods

Data used in the preparation of this article were obtained from the Alzheimer's Disease Neuroimaging Initiative (ADNI) database (www.loni.ucla.edu\ADNI). The ADNI was launched in 2003 by the National Institute on Aging (NIA), the National Institute of Biomedical Imaging and Bioengineering (NIBIB), the Food and Drug Administration (FDA), private pharmaceutical companies and non-profit organizations. The primary goal of ADNI has been to test whether serial magnetic resonance imaging (MRI), positron emission tomography (PET), other biological markers, and clinical and neuropsychological assessment can be combined to measure the progression of mild cognitive impairment (MCI) and early Alzheimer's disease (AD). Determination of sensitive and specific markers of very early AD progression is intended to aid researchers and clinicians to develop new treatments and monitor their effectiveness, as well as lessen the time and cost of clinical trials. Subjects have been recruited from over 50 sites across the U.S. and Canada. The initial goal of ADNI was to recruit 800 adults, ages 55 to 90, to participate in the research—approximately 200 cognitively normal older individuals to be followed for 3 years, 400 people with MCI to be followed for 3 years, and 200 people with early AD to be followed for 2 years.” For up-to-date information see www.adni-info.org.

### Example 3

### Association with CSF Tau/p-Tau₁₈₁ Levels in WU ADRC Series

### Initial Screening

Out of 384 SNPs genotyped, 355 passed the quality control steps (Hardy-Weinberg and call rate >95%). Sixteen samples with a genotype rate lower than 95% were not included in the analyses. Nineteen SNPs showed significant association with CSF tau and ptau₁₈₁ levels in the ADRC series after multiple test correction (Table 5). The most significant SNP, rs1868402, is located in intron 5 of the regulatory subunit B, (PPP3R1) gene (MIM#: 601302), which is a regulatory subunit of the tau-phosphatase PP2B, also called calcineurin. Rs1868402 is associated with CSF tau and ptau₁₈₁ levels in a dominant model, the minor allele is associated with higher CSF levels (p=5.90×10⁻⁰⁴ and 2.25×10⁻⁰⁵ for association with ptau₁₈₁ and tau respectively). Six other SNPs in high linkage disequilibrium (LD) with rs1868402 (FIG. 1), also show association with CSF tau and ptau₁₈₁ levels. Because of the LD in PPP3R1, we only selected rs1868402 and rs6546366 for replication in the ADNI series. The other 12 SNPs that survived multiple test correction were located in phosphatase 3, catalytic subunit, alpha isoform (PPP3CA), cathepsin D (CTSD), coagulation factor II (thrombin, F2), FYN oncogene related to SRC (FYN), GSK3β, MAPT, meningioma expressed antigen 5 (hyaluronidase, MGEA5), O-linked N-acetylglucosamine (GlcNAc) transferase (OGT), protein kinase C, alpha (PRKCA) and WNT1 inducible signaling pathway protein 1 (WISP1) and were also selected for replication (Table 7).

TABLE 7
SNPs associated with CSF tau and ptau₁₈₁ levels in the
initial series and replication in the ADNI samples.

| | | | WU Series | | ADNI series | | Combined Series | |
|---|---|---|---|---|---|---|---|---|
| gene | rs | MAF | Tau | ptau₁₈₁ | Tau | ptau₁₈₁ | Tau | ptau₁₈₁ |
| PPP3R1 | rs1868402^{A} | 0.37 | 2.25 × 10⁻⁰⁵ | 5.90 × 10⁻⁰⁴ | 0.096 | 0.026 | 1.72 × 10⁻⁰⁵ | 5.18 × 10⁻⁰⁵ |
| PPP3R1 | rs6546366^{A} | 0.35 | 2.00 × 10⁻⁰⁴ | 0.004 | 0.284 | 0.488 | 4.57 × 10⁻⁰⁴ | 7.65 × 10⁻⁰³ |
| PPP3CA | rs17030739 | 0.15 | 0.006 | 0.001 | 0.064 | 0.043 | 9.26 × 10⁻⁰⁴ | 2.05 × 10⁻⁰⁴ |
| CTSD | rs1317356^{A} | 0.50 | 0.075 | 0.016 | 0.851 | 0.642 | 0.217 | 0.138 |
| F2 | rs2070852^{A} | 0.32 | 0.002 | 0.008 | 0.602 | 0.948 | 0.007 | 0.051 |
| FYN | rs927010^{B} | 0.26 | 5.00 × 10⁻⁰⁴ | 5.00 × 10⁻⁰⁴ | 0.606 | 0.424 | 0.006 | 0.073 |
| FYN | rs7768046 | 0.39 | 7.00 × 10⁻⁰⁴ | 0.004 | 0.588 | 0.304 | 0.004 | 0.155 |
| GSK3β | rs3755557^{A} | 0.12 | 0.002 | 0.004 | 0.361 | 0.994 | 0.003 | 0.030 |
| GSK3β | rs7431209^{B} | 0.24 | 0.007 | 0.005 | 0.692 | 0.143 | 0.017 | 0.001 |
| MAPT | rs7210728^{A} | 0.36 | 0.004 | 0.005 | 0.776 | 0.440 | 0.114 | 0.008 |
| MGEA5 | rs2305192^{B} | 0.30 | 0.001 | 0.008 | 0.860 | 0.803 | 0.010 | 0.037 |
| OGT | rs6525488 | 0.14 | 5.00 × 10⁻⁰⁴ | 0.023 | 0.598 | 0.451 | 0.029 | 0.236 |
| PRKCA | rs7218425 | 0.21 | 4.60 × 10⁻⁰⁴ | 0.005 | 0.578 | 0.301 | 0.092 | 0.185 |
| WISP1 | rs2930000^{B} | 0.34 | 0.001 | 0.018 | 0.928 | 0.989 | 0.009 | 0.065 |

SNPs passed multiple test correction in the ADRC series were followed up in the ADNI samples. Both series were combined to increase the statistical power.
For each SNP the rs number and P values for association with tau and ptau₁₈₁ are shown.
^{A}dominant model
^{B}Recessive model

### Replication in the ADNI CSF Samples

We used the ADNI CSF samples (Table 4) as a replication series and tested for association with CSF tau/ptau₁₈₁ levels using the same model for which the association was found initially. SNPs located in the genes encoding the regulatory (PPP3R1; rs1868402 p=0.026) and catalytic (PPP3CA; rs17030739; p=0.043) subunits of PP2B showed significant association with ptau₁₈₁ in the same direction and with the same model as was observed in the ADRC series. Rs6546366, also located in PPP3R1 showed no association with CSF ptau₁₈₁ levels in the ADNI series (p=0.284). Rs6546366 showed a lower level of LD with rs1868402 (r²=0.65) than in the ADRC series (r²=0.80), suggesting that rs1868402, and not rs6546366, is the variant that drives the association in PPP3R1.

The ADRC and ADNI CSF series were combined to increase statistical power (Table 7). In the combined series, an association was only considered significant when the p-value was lower than in the ADRC or ADNI series alone. In the combined series, rs1868402 showed the most significant association with CSF tau and ptau₁₈₁ levels (tau P=1.72×10⁻⁰⁵, ptau₁₈₁ p=5.18×10⁻⁰⁵). The SNP located in the catalytic subunit of calcineurin, rs17030739, was also significantly associated with CSF tau (p=9.26×10⁻⁰⁴) and ptau₁₈₁ levels (p=2.05×10⁻⁰⁴). For rs17030739, the major allele is associated with higher CSF levels. Rs7431209, in GSK3β, also showed a more significant association with ptau₁₈₁ in the combined series (p=0.0018), than in the ADRC (p=0.005) or ADNI (p=0.14) series alone.

### Fine Mapping (Imputation) and Haplotype Analyses

Analysis indicated that neither rs1868402 (PPP3R1) nor rs17030739 (PPP3CA) were potentially functional. To identify the functional variant, we investigated whether other SNPs in the PPP3R1 and PPP3CA genes showed a more significant association with CSF ptau₁₈₁ levels by imputing the genotypes for untyped SNPs in a region of 200 kb around each gene. Genotypes for 110 SNPs located in PPP3R1 were successfully imputed, but none of them showed higher association with CSF ptau₁₈₁ levels than rs1868402. For PPP3CA, genotypes were imputed for 282 SNPs. Two SNPs, rs9307252 and rs17030741, in very high LD with rs17030739, showed a slightly more significant association than rs17030739 (Table 8, FIG. 4). Another seven SNPs, in the same LD bin, showed similar p-values (Table 8, FIG. 4). None of the associated SNPs in PPP3CA are predicted to be functional based on analyses performed using the Pupasuite software program. Haplotype analysis of the PPP3R1 and PPP3CA SNPs did not reveal any stronger association than that observed with either rs1868402 or rs17030739 alone. These results suggest that unknown variants in LD with rs1868402 and rs17030739 are responsible for the association observed with CSF tau/ptau₁₈₁ levels.

TABLE 8
PPP3CA imputed SNPs significantly associated with CSF ptau181 levels

| SNP # | p-value |
|---|---|
| rs9307252 | 0.0013 |
| rs17030741 | 0.0013 |
| rs17030739 | 0.0013 |
| rs10020845^{A} | 0.0029 |
| rs7356517^{A} | 0.0029 |
| rs10022217^{A} | 0.0030 |
| rs9993215^{A} | 0.0042 |
| rs10026319^{A} | 0.0042 |
| rs10003855^{A} | 0.0042 |
| rs10026659^{A} | 0.0042 |

For each SNP the rs number and P values, of the best model, for association with ptau₁₈₁ are shown.
^{A}allelic test

### Sample Stratification

In a previous study, we found that SNPs in MAPT were associated with CSF tau levels only in individuals with low CSF Aβ42 levels indicating Aβ deposition in the brain (PNAS). Aβ deposition occurs prior to the occurrence of clinical symptoms due to AD and is thought to represent a preclinical phase of AD. In the ADRC CSF series, individuals with CSF Aβ42 levels less than 500 pg/ml have evidence of Aβ deposition as detected by positron emission tomography using Pittsburgh compound B (PET-PIB) (9). In the ADNI series, the CSF Aβ42 threshold for PIB binding is 192 pg/ml (22). The difference in threshold in the CSF Aβ42 levels for PIB binding between the WU and ADNI series is due to the different antibodies and procedures used to measure the CSF Aβ42 levels, as explained in material and methods. We stratified the WU and ADNI CSF series based on the CSF Aβ42 thresholds to define groups with and without fibrillar Aβ deposition in the brain. In the low CSF Aβ42 group, there are individuals diagnosed with DAT (CDR>0, n=183), but also non-demented individuals (CDR=0, n=134) with likely Aβ deposition in the brain, and brain atrophy (presymptomatic AD) (9, 11, 29).

For the PPP3R1 (rs1868402) and PPP3CA (rs17030739) SNPs, we observed significant association in the low Aβ42 stratum (presence of Aβ deposition in the brain) but not in the high Aβ42 stratum (Table 9). We also observed a gene by gene interaction (rs1868402 p=0.001; rs17030739 p=0.02). Further investigation of the association of rs1868402 in the low Aβ42 stratum demonstrates that the association is stronger in non-demented individuals (CDR 0, p=0.003, n=134; CDR>0 p=0.027, n=183). These results suggest that this SNP, or another SNP in high LD, influences tau-related pathology particularly in the early stages of AD pathogenesis when Aβ pathology has started but neurodegeneration is not high enough to result in clinical symptoms (preclinical AD).

TABLE 9
SNPs are associated with CSF tau and ptau181 levels in
individuals with Aβ deposition

| Gene | Rs # | stratum | n | tau | ptau1 |
|---|---|---|---|---|---|
| PPP3R1 | rs1868402^{A} | Total | 619 | 1.72 × 10⁻⁰⁵ | 5.18 × 10⁻⁰⁵ |
| | | sample | | | |
| | | Low Aβ | 336 | 1.16 × 10⁻⁰⁴ | 1.80 × 10⁻⁰⁴ |
| | | levels | | | |
| | | High Aβ | 283 | 0.049 | 0.096 |
| | | levels | | | |
| PPP3R1 | rs6546366^{A} | Total | 619 | 9.26 × 10⁻⁰⁴ | 2.05 × 10⁻⁰³ |
| | | sample | | | |
| | | Low Aβ | 336 | 1.15 × 10⁻⁰⁴ | 4.68 × 10⁻⁰⁴ |
| | | levels | | | |
| | | High Aβ | 283 | 0.481 | 0.788 |
| | | levels | | | |
| PPP3CA | rs17030739 | Total | 619 | 9.26 × 10⁻⁰⁴ | 2.05 × 10⁻⁰⁴ |
| | | sample | | | |
| | | Low AB | 336 | 7.70 × 10⁻⁰⁵ | 5.76 × 10⁻⁰⁴ |
| | | levels | | | |
| | | High AB | 283 | 0.871 | 0.106 |
| | | levels | | | |

P values for tau and ptau181 in the combine series. Samples were stratified based on CSF Aβ42 levels as an approximation of Aβ deposition. For the ADRC samples individuals with Aβ42 levels less 500 pg/ml were considered positive for Aβ deposition and for ADNI samples the Aβ42 levels associated with Aβ deposition were 193 pg/ml.
Values in boldface indicate P values <0.05.
^{A}Dominant model.

### Example 4

### Gene Expression

We tested whether rs1868402 and rs17030739 are associated with gene expression and tau pathology. None of the SNPs were associated with cDNA levels in the full brain series. However, the minor allele of rs1868402 was associated with increase of tau Braak stage in the full brain series (n=123, p=0.029). Based on our finding in the CSF series, we stratified the brain series by presence of dementia and AD pathology (measured by Braak and Braak stages). As expected, most of the association of rs1868402 with tau pathology was driven by group of non-demented individuals with AD pathology (n=24; p=0.022; FIG. 5). In this stratum, we found that minor alleles carries of rs1868402 also showed lower PPP3R1 mRNA expression, and PPP3R1 mRNA was inversely correlated with tau pathology (measured by Braak stage) (FIG. 5). No association was found between rs17030739 and PPP3CA expression or tau pathology in the full series or strata.

### Example 5

### Risk for AD and Age at Onset

Finally, we tested for association between rs1868402 and rs17030739 and risk for AD or age at onset (AAO). Specifically, we hypothesized that the minor allele of rs1868402, which is associated with higher CSF tau and ptau₁₈₁ levels, lower PPP3R1 mRNA levels and higher plaque/tangle counts, would be increased in AD patients or be associated with earlier age at onset. For rs17030739 the major allele, which is associated with higher CSF ptau₁₈₁ levels, we hypothesized that it would be associated with earlier age at onset or increased risk for AD. We genotyped rs1868402 and rs17030739 in a total of 1204 AD cases and 1227 controls from three different series: ADRC, ADNI and MRC (Table 10). As expected, the minor allele of rs1868402 is increased in AD cases compared to controls in the three series (Table 10). Consistent with the previous results, rs1868402 showed the best fit in the dominant model (AD cases 50.7% vs controls 46.2%; One-tail p: 0.017; OR 1.19 CI95% 1.02-1.39). We did not detect association between rs1868402 and AAO or between rs17030739 and risk for disease or AAO (Table 10 and FIG. 6).

TABLE 10
Case-control analyses for the PPP3R1 and PPP3CA SNPs

| | | | | Minor | MAF | | | |
|---|---|---|---|---|---|---|---|---|
| | Series | Cases | Controls | Allele | Cases | Controls | p-value | OR |
| rs1868402^{A} | MRC | 662 | 801 | C | 0.31 | 0.28 | 0.06 | |
| | ADNI | 230 | 204 | C | 0.28 | 0.26 | 0.03 | |
| | ADRC | 312 | 222 | C | 0.30 | 0.28 | 0.14 | |
| | Total | 1204 | 1227 | C | 0.30 | 0.28 | 0.017 | 1.19 (1.01-1.39) |
| rs17030739 | MRC | 662 | 801 | A | 0.13 | 0.12 | 0.10 | |
| | ADNI | 230 | 204 | A | 0.16 | 0.10 | 0.01 | |
| | ADRC | 312 | 222 | A | 0.13 | 0.15 | 0.12 | |
| | Total | 1204 | 1227 | A | 0.14 | 0.13 | 0.15 | 1.08 (0.93-1.27) |

rs1868402 and rs17030739 were genotyped in the MRC, ADNI and ADRC series. Number of cases and controls, minor allele and minor allele frequency (MAF) for each series and for the combine series are showed.
One-tail p-value for the dominant (rs1868402) or additive (rs1730739) model are showed.
^{A}dominant model

### REFERENCES

1. LaFerla, F. M. & Oddo, S. (2005) Trends Mol Med 11, 170-176.
2. Hansson, O., Zetterberg, H., Buchhave, P., Andreasson, U., Londos, E., Minthon, L., & Blennow, K. (2007) Dement Geriatr Cogn Disord 23, 316-320.
3. Sato-Harada, R., Okabe, S., Umeyama, T., Kanai, Y., & Hirokawa, N. (1996) Cell Struct Funct 21, 283-295.
4. Wang, J. Z., Grundke-Iqbal, I., & Iqbal, K. (2007) Eur J Neurosci 25, 59-68.
5. Spires-Jones, T. L., Stoothoff, W. H., de Calignon, A., Jones, P. B., & Hyman, B. T. (2009) Trends Neurosci 32, 150-159.
6. Kapaki, E. N., Paraskevas, G. P., Tzerakis, N. G., Sfagos, C., Seretis, A., Kararizou, E., & Vassilopoulos, D. (2007) Eur J Neurol 14, 168-173.
7. Fagan, A. M., Roe, C. M., Xiong, C., Mintun, M. A., Morris, J. C., & Holtzman, D. M. (2007) Arch Neurol 64, 343-349.
8. Noguchi, M., Yoshita, M., Matsumoto, Y., Ono, K., Iwasa, K., & Yamada, M. (2005) J Neurol Sci 237, 61-65.
9. Fagan, A. M., Mintun, M. A., Mach, R. H., Lee, S. Y., Dence, C. S., Shah, A. R., LaRossa, G. N., Spinner, M. L., Klunk, W. E., Mathis, C. A., et al. (2006) Ann Neurol 59, 512-519.
10. Ikonomovic, M. D., Klunk, W. E., Abrahamson, E. E., Mathis, C. A., Price, J. C., Tsopelas, N. D., Lopresti, B. J., Ziolko, S., Bi, W., Paljug, W. R., et al. (2008) Brain 131, 1630-1645.
11. Price, J. L. & Morris, J. C. (1999) Ann Neurol 45, 358-368.
12. Hesse, C., Rosengren, L., Andreasen, N., Davidsson, P., Vanderstichele, H., Vanmechelen, E., & Blennow, K. (2001) Neurosci Lett 297, 187-190.
13. Ost, M., Nylen, K., Csajbok, L., Ohrfelt, A. O., Tullberg, M., Wikkelso, C., Nellgard, P., Rosengren, L., Blennow, K., & Nellgard, B. (2006) Neurology 67, 1600-1604.
14. Buerger, K., Ewers, M., Pirttila, T., Zinkowski, R., Alafuzoff, I., Teipel, S. J., DeBernardis, J., Kerkman, D., McCulloch, C., Soininen, H., et al. (2006) Brain 129, 3035-3041.
15. Urakami, K., Wada, K., Arai, H., Sasaki, H., Kanai, M., Shoji, M., Ishizu, H., Kashihara, K., Yamamoto, M., Tsuchiya-Ikemoto, K., et al. (2001) J Neurol Sci 183, 95-98.
16. Arai, H., Morikawa, Y., Higuchi, M., Matsui, T., Clark, C. M., Miura, M., Machida, N., Lee, V. M., Trojanowski, J. Q., & Sasaki, H. (1997) Biochem Biophys Res Commun 236, 262-264.
17. Andersson, C., Blennow, K., Almkvist, O., Andreasen, N., Engfeldt, P., Johansson, S. E., Lindau, M., & Eriksdotter-Jonhagen, M. (2007) Neurobiol Aging.
18. de Leon, M. J., DeSanti, S., Zinkowski, R., Mehta, P. D., Pratico, D., Segal, S., Clark, C., Kerkman, D., DeBernardis, J., Li, J., et al. (2004) J Intern Med 256, 205-223.
19. Morris, J. C., Storandt, M., Miller, J. P., McKeel, D. W., Price, J. L., Rubin, E. H., & Berg, L. (2001) Arch Neurol 58, 397-405.
20. Kauwe, J. S., Cruchaga, C., Mayo, K., Fenoglio, C., Bertelsen, S., Nowotny, P., Galimberti, D., Scarpini, E., Morris, J. C., Fagan, A. M., et al. (2008) Proc Natl Acad Sci USA 105, 8050-8054.
21. Morris, J. C. (1993) Neurology 43, 2412-2414.
22. Shaw, L. M., Vanderstichele, H., Knapik-Czajka, M., Clark, C. M., Aisen, P. S., Petersen, R. C., Blennow, K., Soares, H., Simon, A., Lewczuk, P., et al. (2009) Ann Neurol.
23. Braak, H. & Braak, E. (1991) Acta Neuropathol (Berl) 82, 239-259.
24. Morgan, A. R., Turic, D., Jehu, L., Hamilton, G., Hollingworth, P., Moskvina, V., Jones, L., Lovestone, S., Brayne, C., Rubinsztein, D. C., et al. (2007) Am J Med Genet B Neuropsychiatr Genet 144B, 762-770.
25. McKhann, G., Drachman, D., Folstein, M., Katzman, R., Price, D., & Stadlan, E. M. (1984) Neurology 34, 939-944.
26. Stoothoff, W. H. & Johnson, G. V. (2005) Biochim Biophys Acta 1739, 280-297.
27. Zachara, N. E. & Hart, G. W. (2006) Biochim Biophys Acta 1761, 599-617.
28. Muller, P. Y., Janovjak, H., Miserez, A. R., & Dobbie, Z. (2002) Biotechniques 32, 1372-1374, 1376, 1378-1379.
29. Fagan, A. M., Head, D., Shah, A. R., Marcus, D., Mintun, M., Morris, J. C., & Holtzman, D. M. (2009) Ann Neurol 65, 176-183.
30. Yu, D. Y., Luo, J., Bu, F., Song, G. J., Zhang, L. Q., & Wei, Q. (2006) Biological chemistry 387, 977-983.
31. Luo, J., Ma, J., Yu, D. Y., Bu, F., Zhang, W., Tu, L. H., & Wei, Q. (2008) Brain Res Bull 76, 464-468.
32. Garver, T. D., Kincaid, R. L., Conn, R. A., & Billingsley, M. L. (1999) Molecular pharmacology 55, 632-641.
33. Kayyali, U.S., Zhang, W., Yee, A. G., Seidman, J. G., & Potter, H. (1997) J Neurochem 68, 1668-1678.
34. Ladner, C. J., Czech, J., Maurice, J., Lorens, S. A., & Lee, J. M. (1996) J Neuropathol Exp Neurol 55, 924-931.

1. A biomarker for a neurodegenerative disorder, the biomarker comprising at least one polymorphism in a nucleotide sequence selected from the group consisting of PPP3R1, GSK3β, PPP3CA, FYN, WISP1, MGEA5, CTSD, F2, MAPT, OGT and PRKCA, wherein the polymorphism shows linkage disequilibrium and has a correlation value of greater than about 0.7 when compared to a polymorphism in a nucleotide sequence associated with a neurodegenerative disorder.

2. The biomarker of claim 1, wherein the polymorphism is a single nucleotide polymorphism (SNP) selected from the group consisting of rs1060842, rs1868402, rs4671880, rs12713636, rs13028330, rs10208241, rs6546366, rs7431209, rs17030739, rs927010, rs7768046, rs2930000, rs2305192, rs7218425, rs1317356, rs2070852, rs7210728, rs6525488, rs9307252, rs17030741, rs9993215, rs10026319, rs10003855, rs10026659, rs10022217, rs10020845, rs7356517, rs9307252, rs17232534, rs17030741, and combinations thereof.

3. The biomarker of claim 2, wherein the nucleotide sequence is PPP3R1 and the SNP is selected from the group consisting of rs1060842, rs1868402, rs4671880, rs12713636, rs13028330, rs10208241, rs6546366, and combinations thereof.

4. (canceled)

5. The biomarker of claim 2, wherein the nucleotide sequence is PPP3CA and the SNP is selected from the group consisting of rs9993215, rs10026319, rs10003855, rs10026659, rs10022217, rs10020845, rs7356517, rs17030739, rs9307252, rs17232534, rs17030741, and combinations thereof.

6. (canceled)

7. The biomarker of claim 2, wherein the SNP is associated with the level of tau protein and/or phosphorylated tau protein in a subject.

8. The biomarker of claim 1, wherein the neurodegenerative disorder comprises a tauopathy selected from the group consisting of Alzheimer's disease, corticobasal degeneration, Down's syndrome, frontotemporal dementia with Parkinsonism linked to chromosome 17, Pick's disease, progressive supranuclear palsy, sporadic frontotemporal dementia, and subacute sclerosing panencephalitis.

9. (canceled)

10. A method for identifying a subject at risk for a neurodegenerative disorder, the method comprising determining the identity of at least one polymorphism in the subject in a nucleotide sequence selected from the group consisting of PPP3R1, GSK3β, PPP3CA, FYN, WISP1, MGEA5, CTSD, F2, MAPT, OGT and PRKCA, the polymorphism showing linkage disequilibrium and having a correlation value of greater than about 0.7 when compared to a polymorphism in a nucleotide sequence associated with a neurodegenerative disorder, wherein the presence of one allele of the polymorphism is associated with increased risk for the neurodegenerative disorder.

11. The method of claim 10, wherein the polymorphism is a single nucleotide polymorphism (SNP) selected from the group consisting of rs1060842, rs1868402, rs4671880, rs12713636, rs13028330, rs10208241, rs6546366, rs7431209, rs17030739, rs927010, rs7768046, rs2930000, rs2305192, rs7218425, rs1317356, rs2070852, rs7210728, rs6525488, rs9307252, rs17030741, rs9993215, rs10026319, rs10003855, rs10026659, rs10022217, rs10020845, rs7356517, rs9307252, rs17232534, rs17030741, and combinations thereof.

12. The method of claim 11, wherein the nucleotide sequence is PPP3R1 and the SNP is selected from the group consisting of rs1060842, rs1868402, rs4671880, rs12713636, rs13028330, rs10208241, rs6546366, and combinations thereof.

13. (canceled)

14. (canceled)

15. The method of claim 11, wherein the nucleotide sequence is PPP3CA and the SNP is selected from the group consisting of rs9993215, rs10026319, rs10003855, rs10026659, rs10022217, rs10020845, rs7356517, rs17030739, rs9307252, rs17232534, rs17030741, and combinations thereof.

16. (canceled)

17. (canceled)

18. The method of claim 11, wherein the SNP is associated with the level of tau protein and/or phosphorylated tau protein in the subject.

19. The method of claim 10, wherein the neurodegenerative disorder comprises a tauopathy selected from the group consisting of Alzheimer's disease, corticobasal degeneration, Down's syndrome, frontotemporal dementia with Parkinsonism linked to chromosome 17, Pick's disease, progressive supranuclear palsy, sporadic frontotemporal dementia, and subacute sclerosing panencephalitis.

20-35. (canceled)

36. A kit for SNP genotyping a subject, the kit comprising at least one allele specific oligonucleotide that is complementary to a single nucleotide polymorphism (SNP) nucleic acid, the SNP nucleic acid being selected from the group consisting of SEQ ID NOs:1-28.

37. The kit of claim 36, wherein the oligonucleotide is complementary to one allele of the SNP and from about 7 to about 15 contiguous nucleotides on each side of the SNP.

38-39. (canceled)

40. The kit of claim 36, wherein a first oligonucleotide is complementary to the major allele of the SNP and a second oligonucleotide is complementary to the minor allele of the SNP.

41. The kit of claim 36, wherein the oligonucleotide is attached to a solid support selected from the group consisting of a microarray and a bead.

42. The kit of claim 36, wherein the oligonucleotide further comprises at least one moiety selected from the group consisting of a fluorophore, a quencher, a luminescent chelate, a biotin molecule, and a radioisotope.

43. The kit of claim 36, wherein the oligonucleotide further comprises additional nucleotides with no complementarity to SNP nucleic acid.

44. (canceled)

45. The method of claim 10, wherein the risk is an increased risk for an earlier age at onset of a neurodegenerative disorder.

46. The method of claim 10, wherein the risk is an increased risk for rapid progression of a neurodegenerative disorder.
